(19) United States
(12) Patent Application Publication
(10) Pub. No.: US 2010/0209359 A1
(43) Pub. Date: Aug. 19, 2010

(54) MEDICATED CHEWING GUM

(75) Inventors: Beth M Foster, Bristol (GB); Hongli Yang, Ealing (GB); Terence Cosgrove, Portishead (GB); Erol A. Hasan, Liverpool (GB)

Correspondence Address: OLIFF & BERRIDGE, PLC, P.O. BOX 320850, ALEXANDRIA, VA, 22320-4850, US

(73) Assignee: REVOLYMER LIMITED, London (GB)

(21) Appl. No.: 12/449,628

(22) PCT Filed: Feb. 26, 2008
(86) PCT No.: PCT/EP2008/052326

§ 371 Date: Apr. 12, 2010

(30) Foreign Application Priority Data
Feb. 26, 2007 (EP) 07103052.2
Oct. 15, 2007 (EP) 07118487.3
Nov. 26, 2007 (EP) 07121564.4

(51) Int. Cl. A61K 9/68; A23G 4/08; A23G 4/18; A61P 29/00; A61P 25/00; A61P 31/04; A61P 37/08; A61P 1/08; A61P 35/00; A61P 9/10; A61P 1/04; A61P 15/00

(52) U.S. Cl. 424/48; 426/3; 426/6; 426/5

(57) ABSTRACT

The present invention provides a chewing gum composition comprising a chewing gum base, a biologically active ingredient, a polymeric material and one or more sweetening and flavouring agents, wherein the polymeric material is amphiphilic, has a straight or branched chain carbon-carbon backbone and a multiplicity of side chains attached to the backbone. A method of making the chewing gum composition is also provided.

### STATEMENT OF INVENTION

The present invention relates to a chewing gum composition comprising a chewing gum base, a biologically active ingredient and one or more flavouring or sweetening agents. Methods for preparing the chewing gum compositions are also provided.

### BACKGROUND OF INVENTION

Chewing gum compositions typically comprise a water-soluble bulk portion, a water insoluble chewable gum base and flavouring agents. The gum base typically contains a mixture of elastomers, vinyl polymers, elastomer solvents or plasticisers, emulsifiers, fillers and softeners (plasticisers). The elastomers, waxes, elastomer solvents and vinyl polymers are all known to contribute to the gum base's adhesiveness.

Biologically active ingredients have been previously incorporated into chewing gum compositions. Morjaria et al in Dissolution Technologies, May 2004, 12-15, in an article entitled “In Vitro Release of Nicotine from Chewing Gum Formulations” evaluate the release of nicotine from conventional gums using the European Pharmacopoeia apparatus. The release profiles are compared to Pharmagu M®, a compactable gum that has been developed by SPI Pharma.

WO 00/35298 describes a chewing gum containing medicament active agents. The release of the active agent is controlled by physically modifying the active agent by coating and drying. Chewing gum with the active ingredients caffeine, nicotine, ibuprofen, ketoprofen and naproxen are all specifically mentioned.

Nicorette™ is a well-known example of a marketed chewing gum comprising nicotine.

U.S. Pat. No. 6,592,850 describes a chewing gum containing sildenafil citrate which may be used to treat erectile dysfunction. In the manufacturing method described in this Patent, the medicament is mixed with gum base, sweetener and a flavouring agent, preferably within the first 5 minutes of mixing.

In view of the prior art, there is a need to provide improved chewing gum compositions for the delivery of biologically active ingredients, such as nicotine, to the body.

### SUMMARY OF INVENTION

In accordance with a first aspect of the invention there is provided a chewing gum composition comprising a chewing gum base, a biologically active ingredient, a polymeric material and one or more sweetening or flavouring agents, wherein the polymeric material is amphiphilic, has a straight or branched chain carbon-carbon backbone and a multiplicity of side chains attached to the backbone.

In the chewing gum composition according to the first aspect of this invention the release of the biologically active ingredient is controlled. The nature and strength of the interaction between the active ingredient and polymeric material determines whether the active is released quickly or exhibits delayed-release. It has been shown that the polymeric material may also influence the total amount of active released, in some cases, releasing more active than gums of the prior art over a set time period. This means that less active can be used in the chewing gum compositions of this invention, compared to those in the prior art.

By chewing the chewing gum composition of the present invention the active is released from the chewing gum. Saliva coats the oral tissues under the tongue (sublingual) and the sides of the mouth where the drug may partition from the saliva into the oral mucosa. It is thought that chewing creates pressure in the buccal cavity which forces the active ingredient directly into the systemic system of the individual through the oral mucosa contained in the buccal cavity. This greatly accelerates absorption of the drug into the systemic system, compared to the typical gastro-intestinal routes.

In accordance with a second aspect of this invention, there is provided a method of forming a chewing gum composition comprising the steps of (i) forming a chewing gum base by mixing an elastomeric material optionally with one or more elastomer plasticisers, softeners, fillers, emulsifiers and waxes; (ii) adding the biologically active ingredient to the gum base, together with one or more sweetening or flavouring agents, to form a chewing gum composition;

wherein a polymeric material which is amphiphilic and has a straight or branched chain carbon-carbon backbone and a multiplicity of side chains attached to the backbone is added to the chewing gum base in step (i) and/or to the chewing gum composition in step (ii).

In step (i), the chewing gum base is formed by mixing typical gum base components known in the art. These typically include elastomeric material and optionally one or more of the following: elastomer plasticisers, softeners, fillers, emulsifiers and waxes, as described in more detail below.

This method has been shown to provide stable chewing gum compositions with uniform distribution of drug and excellent chewability.

We have disclosed in our previous Patent Application, published as WO 2006/016179, that the polymeric materials defined above have reduced tack, and may reduce the adhesiveness of chewing gum compositions. The polymeric materials have a straight or branched chain carbon-carbon polymer backbone, and a multiplicity of side chains attached to the backbone. The side chains are derived from an alkylsilyl polyoxyalkylene or a polyoxyalkylene. This is the first time that use of these polymers to control the release of biologically active ingredients has been described.

### PREFERRED EMBODIMENTS OF THE INVENTION

### Gum Base

Typically, the chewing gum base comprises 2-90% by weight of the amphiphilic polymeric material, preferably, 2-50%, more preferably 2-25%, most preferably 3-20% by weight. The polymeric material may act as a substitute for part or ail of the ingredients in the gum base which contribute to adhesiveness.

Alternatively, the gum base comprises no amphiphilic polymeric material. Instead, the amphiphilic material is added to the chewing gum composition independently of the chewing gum base. Most typically, the amphiphilic polymer is added to both the gum base and chewing gum composition.

The chewing gum base may comprise 0-6% by weight wax. Examples of waxes which may be present in the gum base include microcrystalline wax, natural wax, petroleum wax, paraffin wax and mixtures thereof. Waxes normally aid in the solidification of gum bases and improving the shelf-life and texture. Waxes have also been found to soften the base mixture, improve elasticity during chewing and affect flavour retention. Preferably, the gum base comprises substantially no wax, and these properties are provided by the polymeric material. However, in some embodiments wax is present and this works with the amphiphilic polymer to control the release of the active.

The elastomeric material provides desirable elasticity and textural properties as well as bulk. Suitable elastomeric materials include synthetic and natural rubber. More specifically, the elastomeric material is selected from butadiene-styrene copolymers, polyisobutylene and isobutylene-isoprene copolymers. It has been found that if the total amount of elastomeric material is too low, the gum base lacks elasticity, chewing texture and cohesiveness, whereas if the content is too high, the gum base is hard and rubbery. Typical gum bases contain 10-70% by weight elastomeric material, more typically 10-15% by weight. Typically, the polymeric material will form at least 1% by weight, preferably at least 10% by weight, more preferably at least 50% by weight of the elastomeric material in the chewing gum base. In some embodiments, the polymeric material completely replaces the elastomeric material in the chewing gum base.

Elastomer plasticisers (also known as elastomer solvents) aid in softening the elastomeric material and include methyl glycerol or pentaerythritol esters of rosins or modified rosins, such as hydrogenated, dimerized, or polymerized rosins or mixtures thereof. Examples of elastomer plasticisers suitable for use in the chewing gum base of the present invention include the pentaerythritol ester of partially hydrogenated wood rosin, pentaerythritol ester of wood rosin, glycerol ester of partially dimerized rosin, glycerol ester of polymerised rosin, glycerol ester of tall oil rosin, glycerol ester of wood rosin and partially hydrogenated wood rosin and partially hydrogenated methyl ester of rosin; terpene resins including polyterpene such as d-limonene polymer and polymers of α-pinene or β-pinene and mixtures thereof. Elastomer plasticisers may be used up to 30% by weight of the gum base. The preferred range of elastomer solvent, however, is 2-18% by weight. Preferably it is less than 15% by weight. Alternatively, no elastomer solvent may be used.

The weight ratio of elastomer plus polymeric material to elastomer plasticiser is preferably in the range (1 to 50):1 preferably (2 to 10):1.

The chewing gum base preferably comprises a non-toxic vinyl polymer. Such polymers may have some affinity for water and include poly(vinyl acetate), ethylene/vinyl acetate and vinyl laurate/vinyl acetate copolymers. Preferably, the non-toxic vinyl polymer is poly(vinyl acetate). Preferably, the non-toxic vinyl polymer is present at 15-45% by weight of the chewing gum base. The non-toxic vinyl polymer should have a molecular weight of at least 2000. Unless otherwise specified, the unit of molecular weight used in this specification is g/mol.

In alternative embodiments, the chewing gum base comprises no vinyl polymer.

The chewing gum base preferably also comprises a filler, preferably a particulate filler. Fillers are used to modify the texture of the gum base and aid in its processing. Examples of typical fillers include calcium carbonate, talc, amorphous silica and tricalcium phosphate. Preferably, the filler is silica, or calcium carbonate. The size of the filler particle has an effect on cohesiveness, density and processing characteristics of the gum base on compounding. Smaller filler particles have been shown to reduce the adhesiveness of the gum base.

The amount of filler present in the chewing gum base is typically 0-40% by weight of the chewing gum base, more typically 5-15% by weight.

Preferably, the chewing gum base comprises a softener. Softeners are used to regulate cohesiveness, to modify the texture and to introduce sharp melting transitions during chewing of a product. Softeners ensure thorough blending of the gum base. Typical examples of softeners are hydrogenated vegetable oils, lanolin, stearic acid, sodium stearate, potassium stearate and glycerine. Softeners are typically used in amounts of about 15% to about 40% by weight of the chewing gum base, and preferably in amounts of from about 20% to about 35% of the chewing gum base.

A preferred chewing gum base comprises an emulsifier. Emulsifiers aid in dispersing the immiscible components of the chewing gum composition into a single stable system. Suitable examples are lecithin, glycerol, glycerol monooleate, lactylic esters of fatty acids, lactylated fatty acid esters of glycerol and propylene glycol, mono-, di-, and tri-stearyl acetates, monoglyceride citrate, stearic acid, stearyl monoglyceridyl citrate, stearyl-2-lactylic acid, triacyetyl glycerin, triethyl citrate and polyethylene glycol. The emulsifier typically comprises from about 0% to about 15%, and preferably about 4% to about 6% of the chewing gum base.

The backbone of the polymeric material used in the chewing gum base according to the present invention is preferably derived from a homopolymer of an ethylenically unsaturated hydrocarbon monomer or from a copolymer of two or more ethylenically unsaturated hydrocarbon monomers. The base polymers from which the polymeric material is derived, i.e. without the side chains, is an elastomeric material. The polymeric material as a whole may also be an elastomeric material.

The amphiphilic polymeric material has a carbon-carbon polymer backbone typically derived from a homopolymer of an ethylenically-unsaturated polymerisable hydrocarbon monomer or from a copolymer of two or more ethylenically-unsaturated polymerisable hydrocarbon monomers. By the term “ethylenically-unsaturated polymerisable hydrocarbon monomer” we mean a polymerisable hydrocarbon containing at least one carbon-carbon double bond which is capable of undergoing addition (otherwise known as chain-growth or chain-reaction) polymerisation to form a straight or branched chain hydrocarbon polymer having a carbon-carbon polymer backbone. According to one preferred embodiment, the carbon-carbon polymer backbone is derived from a homopolymer of an ethylenically-unsaturated polymerisable hydrocarbon monomer containing 4 or 5 carbon atoms, for example, isobutylene (2-methylpropene). The carbon-carbon polymer backbone may also, according to another embodiment, be derived from a homopolymer of a conjugated diene hydrocarbon monomer, especially one containing 4 or 5 carbon atoms, such as 1,3-butadiene or isoprene.

As mentioned above, the carbon-carbon polymer backbone may be derived from a copolymer of two or more ethylenically-unsaturated polymerisable hydrocarbon monomers. Preferably, it is derived from a copolymer of two such monomers. For example, it may be derived from a hydrocarbon copolymer of a hydrocarbon monomer having one carbon-carbon double bond and a hydrocarbon monomer having two carbon-carbon double bonds. For example, the carbon-carbon polymer backbone may be derived from a copolymer of isobutylene and isoprene. According to a different embodiment, the carbon-carbon polymer backbone is derived from a butadiene-styrene block copolymer. The backbone may be random, alternating or block, e.g. A-B or AB-A block, copolymers.

Alternatively, the amphiphilic polymeric material has a backbone which is a copolymer of at least one ethylenically-unsaturated monomer and maleic anhydride. The term copolymer covers both bipolymers and terpolymers. Preferably the monomer is a hydrocarbon monomer. By the term “ethylenically-unsaturated polymerisable hydrocarbon monomer” we mean a polymerisable hydrocarbon containing at least one carbon-carbon double bond which is capable of undergoing polymerisation to form a straight or branched chain hydrocarbon polymer having a carbon-carbon polymer backbone. According to one preferred embodiment, the ethylenically-unsaturated polymerisable hydrocarbon monomer contains 4 or 5 carbon atoms, and is, for instance, isobutylene (2-methylpropene). The ethylenically unsaturated monomer may alternatively be a conjugated diene hydrocarbon monomer, especially one containing 4 or 5 carbon atoms, such as 1,3-butadiene or isoprene. The ethylenically-unsaturated monomer may alternatively be 1-octadecene.

In this aspect of the invention, the ethylenically unsaturated monomer may be aromatic and/or contains atoms other than hydrogen and carbon. Suitable ethylenically unsaturated monomers include styrene and vinyl methyl ether.

The hydrocarbon polymer, from which the backbone of the polymeric material is derived, typically has a molecular weight in the range 10,000 to 200,000, preferably 15,000 to 50,000, more preferably from 25,000 to 45,000.

The backbone of the polymeric material is typically hydrophobic in nature. In contrast, the side chains may be hydrophilic, which confer several advantages. The hydrophobic/hydrophilic balance of the comb-like copolymer structure leads to a substantial change in the hardness of the gum base in the dry state, making the discarded cud easier to remove from surfaces. Furthermore, hydrophilic side chains may allow saliva to act as an elastomer solvent on chewing, making the gum more chewable. This advantageously allows some or all of the wax and/or elastomer solvent content to be replaced by the polymeric material.

The hydrophilic side chains confer surface active properties on the polymeric material. In the gum base the polymeric material with hydrophilic side chains becomes surface enriched during chewing, giving a hydrophilic coating which does not bind to hydrophobic surfaces, such as asphalts and greasy paving stones. In the presence of water the polymeric material is more easily removable from the most common surfaces.

Furthermore, the amphiphilic nature of the polymeric material allows favourable interactions between the material and the biologically active ingredient, allowing the ingredient to be incorporated into the chewing gum composition, and released during chewing of the gum in the mouth.

The hydrophilic side chains of the polymeric material are preferably derived from poly(ethylene oxide), polyglycidol, poly(vinyl alcohol), poly(styrene sulphonate) or poly(acrylic acid), most preferably poly(ethylene oxide). Poly(ethylene oxide) binds strongly to simple anionic surfactants such as those used in hair shampoo and washing up liquids, to make an electrolyte. In the presence of such anionic surfactants and water, the polymeric material is repelled by most common anionic surfaces which includes many oxide surfaces, cotton clothing and hair. This advantageously allows the gum base to be removed by washing with soapy water.

Alternatively, the side chains may be derived from a polypeptide, for example polylysine.

Alternatively, the side chains of the polymeric material may be more hydrophobic than the backbone. Suitable examples include fluoroalkanes, polysilanes, polyalkylsilanes, alkylsilyl polyoxyalkylenes and siloxanes, which impart a very low surface energy to the gum base.

Each backbone of polymeric material may have a plurality of side chains which may include a mixture of the side chains listed above, and/or have different chain lengths/molecular weights. Preferably, however, each side chain has the same chain length/molecular weight.

The chewing gum base or composition may comprise two or more of the polymeric materials discussed above.

Preferably, the side chains of the polymeric material have the formula

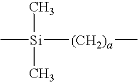

or have the formula

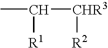

wherein R¹ is H, —C(O)OR⁴ or —C(O)Q and R² is —C(O)OR⁴ or —C(O)Q provided that at least one of R¹ and R² is the group —C(O)Q;
R³ is H or —CH₃;
R⁴ is H or an alkyl group having from 1 to 6 carbon atoms;
Q is a group having the formula —O—(YO)_{b}—(ZO)_{c}—R⁵, wherein each of Y and Z is, independently, an alkylene group having from 2 to 4 carbon atoms and R⁵ is H or an alkyl group having from 1 to 4 carbon atoms;
a is 3 or 4, and each of b and c is, independently, 0 or an integer of from 1 to 125 provided that the sum b+c has a value in the range of from 10 to 250, preferably from 10 to 120.

Preferably, the side chains are attached to the backbone of the polymeric material via a group derived from maleic anhydride.

According to one embodiment of the present invention, the side chains in the polymeric material have the formula

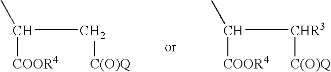

wherein R³, R⁴ and Q are as defined above. These groups are derived from maleic anhydride units or derivatives thereof grafted onto the backbone.

Preferably, the polymeric material has pendant carboxylic acid groups. In the above formula therefore, preferably R⁴ is H.

According to another embodiment, the side chains may have formula

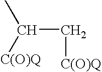

wherein Q is as defined above.

In another embodiment the side chains have the following formula

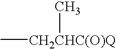

wherein Q is as defined above. These are derived from methacrylic-grafted materials.

According to another embodiment the side chains may have the formula

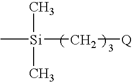

Alternatively, the side chains may have formula

—CH₂CH₂C(O)Q

These are derived from acrylic grafted materials.

Two polymeric materials which may be used in the novel chewing gum base are detailed in Table 1 below. Two partially preferred polymeric materials are P1 and P2.

TABLE 1
Polymeric materials

| Name | Backbone Starting Material | Side Chains |
|---|---|---|
| P1 | PIP-g-MA | PEO 2K |
| P2 | PIP-g-MaMme | PEO 2K |

PIP = polyisoprene;
g = graft;
MA = maleic anhydride;
MaMme = Monoacid monomethyl ester;
PEO = polyethylene oxide and
K = 1000 molecular weight units.

Any PIP-g-MA of appropriate molecular weight distribution and maleic anhydride content will be suitable for the synthesis of the graft copolymer. Alternatively carboxylated PIP-g-MA materials in which the maleic anhydride is ring opened to form a diacid or mono-acid/mono-methyl ester will also be suitable, the latter is demonstrated in P2.

The backbones of each of these polymers are derived from polyisoprene to which maleic anhydride has been grafted. The level of grafting of MA is typically around 1.0 mol % in the PIP-g-MA used to demonstrate the concept. In PIP-g-MaMme the same level was 2.7 mol % of the mono-acid mono-methyl ester of MA. The level of grafting depends on the degree of functionalisation of the polyisoprene. For example, in P1 the number of grafts per chain is generally between 1 and 7, whereas in P2 it is between 1 and 10.

It is possible, by varying the alkyleneoxy side chain length, to produce a polymeric material having the desired balance of elastomeric and hydrophilic properties. Increasing the alkyleneoxy chain length increases the hydrophilic nature of the polymeric material. The multipliers b and c in the group Q above are each independently from 0 to 125 provided that the sum b+c lies within the range of from 10 to 250. Preferably b+c is in the range of from 10 to 120, more preferably 20 to 60, especially from 30 to 50 and most especially from 40 to 45. This imparts to the polymer the requisite degree of hydrophilicity.

It is not necessary for ail of the side chains to share the same value of b and c.

Since the hydrophobicity in the side chains increases with carbon content, it is preferred that both Y and Z are ethylene groups. Similarly, in order to not detract from the hydrophilic nature of the side chains, R⁵ is preferably H or CH₃.

As stated above, the properties of the polymeric material depend not only on the character of the side chains grafted onto the carbon-carbon polymer backbone but also on the number of grafted side chains. It is essential according to the invention that a multiplicity of side chains are attached to the backbone. The term “multiplicity” is defined herein as meaning one or more grafted side chains. The number of side chains grafted onto the carbon-carbon polymer backbone, according to the present invention, will typically be an average of at least one side chain on the carbon-carbon polymer backbone. The actual number of side chains grafted onto the carbon-carbon polymer backbone depends on the identity of the side chain and the method by which the side chain is grafted onto the polymer backbone (and the reaction conditions employed therein). In order to achieve a desired degree of hydrophilicity in the polymeric material, it is preferred that the ratio of side chains to backbone units is in the range 1:350 to 1:20, but more preferably 1:100 to 1:30. The side chains are typically statistically disturbed along the carbon-carbon polymer backbone since the location of attachment of the side chain on the backbone will depend on the positions of suitable attachment locations in the backbone of the hydrocarbon polymer used in the manufacture.

When the side chains are linked to the polymer backbone via grafted maleic anhydride units, each maleic anhydride unit in the polymer backbone may be derivatised with either zero, one or two side chains.

In one embodiment of the invention, each side chain has two groups whereby it may be linked to two backbones, thereby forming a cross-linked structure. For instance, a polyethylene glycol side chain is generally terminated with an alcohol at each end, before derivatisation. Each alcohol may be grafted onto a backbone maleic anhydride unit.

A preferred polymeric material used in the gum base according to the present invention has side chains, attached directly to carbon atoms in the carbon-carbon polymer backbone, wherein the side chains have the formula:

—CH₂CH(CH₃)—C(O)—O—(YO)_{b}—(ZO)_{c}—R⁵;

in which Y, Z, R⁵, b and c are as defined above may be prepared by a method which comprises reacting a straight or branched chain hydrocarbon polymer, in a solvent and in an inert atmosphere, with the monomethacrylate compound:

CH₂═C(CH₃)C(O)O—(YO)_{b}—(ZO)_{c}—R⁵;

in the presence of a free radical initiator. The reaction between the hydrocarbon polymer and the methacrylate compound is carried out as further described in WO2006/016179.

A polymeric material according to the present invention wherein the side chains, attached directly to carbon atoms in the carbon-carbon polymer backbone, have the formula

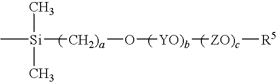

in which Y, Z, R⁵, a, b and c are as defined above, may be prepared by a method which comprises
(i) reacting a compound of the formula

HO—(YO)_{b}—(ZO)_{c}—R⁵

with sodium hydride in a dry organic solvent under inert atmosphere;
(ii) reacting the product from step (I) with the compound

CH₂═CH—(CH₂)_{q}—Br,
where q is 1 or 2,
to give the compound II

CH₂═CH—(CH₂)_{q}—O—(YO)_{b}—(ZO)_{c}—R⁵ II
(iii) reacting the compound II with chlorodimethylsilane to give the compound III

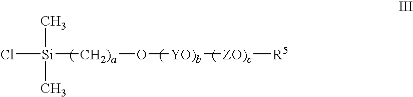

and
(iv) reducing compound III and reacting the product α-hydrodimethylsilyl polyalkylene oxide with a straight or branched chain hydrocarbon polymer containing a multiplicity of carbon-carbon double bonds in the hydrocarbon polymer backbone in the presence of a transition metal salt.

Preferably, in step (ii) above, the product from step (i) is reacted with 3-bromopropene such that, in the formula given above for the side chain, a is 3.

The process is disclosed further in WO2006/016179.

A polymeric material according to the present invention wherein the side chains, attached directly to carbon atoms in the carbon-carbon polymer backbone, have the formula

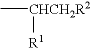

in which one of R¹ and R² is —C(O)Q and the other is —C(O)OR⁴, where Q and R⁴ are as defined above, may be made by a method which comprises reacting polyisoprene-graft-maleic anhydride or a monoester derivative thereof with the compound HO—(YO)_{b}—(ZO)_{c}—R⁵, in which Y, Z, R⁵, b and c are as defined above. Typically, the reaction is carried out in an organic solvent such as toluene.

In the method described above, the number of side chains attached to the polymer backbone will depend on the number of maleic anhydride grafts on the polyisoprene molecule which can take part in the esterification reaction with the alcohol HO—(YO)_{b}—(ZO)_{c}—R⁵. For instance, using a polyisoprene-graft-maleic anhydride of the formula

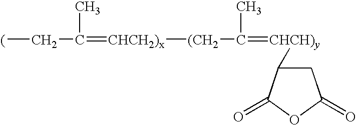

the number of side chains having the general formula given above that can be formed will obviously depend on the value of y. Polyisoprene-graft-maleic anhydride (PIP-g-MA) is available commercially. Purely by way of example one such PIP-g-MA, having the CAS No. 139948-75-7, available from the company, Aldrich, has an average molecular weight of about 25,000. The monomer ratio of isoprene units to maleic anhydride units in this graft copolymer is typically 98:1.1 which indicates that the reaction between this PIP-g-MA and the alcohol described above could produce approximately between 1 and 7 side chains per molecule. Polyisoprene-graft-maleic anhydride may be prepared according to techniques describe in the literature. For instance, according to Visonte L. L. Y. et al, Polymers for Advanced Technologies, Vol 4, 1993, pp 490-495, polyisoprene, dissolved in o-dichlorobenzene, was reacted with maleic anhydride at 180-190° C. to give the modified isoprene. Various polyisoprene-g-maleic anhydride copolymers with 7, 15, 19, 26 and 29 mol % maleic anhydride were obtained by increasing the reaction time from 5 to 11 hours.

The reaction between the PIP-g-MA and the poly(alkyleneoxy) alcohol is typically carried out in an organic solvent such as toluene and typically in the presence of an activator, for example, triethylamine at elevated temperature. The yield of the ester, in this reaction, may be increased by removal of the water from the reaction mixture by azeotropic distillation since toluene and water form azeotropic mixtures which boil at a lower temperature than any of the components. The poly(alkyleneoxy) alcohol may also be reacted with a monoester derivative of PIP-g-MA. For instance, we have achieved good results using a monomethyl ester with the general formula

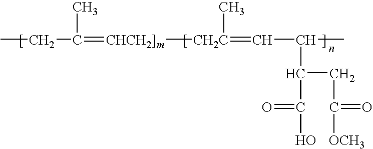

and has a functionality (i.e. n) of approximately 10, an average molecular weight of about 25,000, and a glass transition temperature of −59° C. The reaction of this monomethyl ester with the poly(alkylene oxy) alcohol is typically carried out in an organic solvent such as toluene at an elevated temperature. The yield of ester may be increased by removing water from the reaction mixture by azeotropic distillation. Alternatively the reaction may be performed without solvent, by mixing a melt of either polyisoprene backbone with that of the poly(alkylene oxy) alcohol graft. These methods, although they require the use of preformed polyisoprene having carboxy functionality, have the advantage that they involve relatively simple and quick reactions and give high yields.

When the backbone of the amphiphilic polymeric material is a copolymer of maleic anhydride together with an ethylenically-unsaturated monomer, side chain precursors are typically terminated by an alcohol unit at one end and an alkyloxy group at the other. MeO-PEO-OH is an example of a preferred side chain precursor. In the method of formation of the polymeric material such side chains react with the maleic anhydride derived units via alcoholysis of the anhydride to give a carboxylic ester and carboxylic acid.

The reaction of maleic anhydride with an alcohol is an alcoholysis reaction which results in the formation of an ester and a carboxylic acid. The reaction is also known as esterification. The reaction is relatively fast and requires no catalyst, although acid or base catalysts may be used.

The net reaction may be represented as shown below. Pₓ and P_{Y} represent the remainder of the copolymer/terpolymer and ROH is a representative side chain precursor.

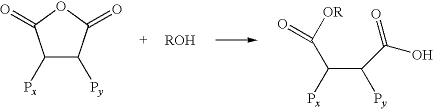

In the method two side chains precursors represented by ROH may react at the same maleic anhydride monomer to give a compound of general formula

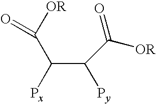

Alternatively, only one side chain precursor reacts per maleic anhydride monomer. This leaves the unit derived from maleic anhydride with a free carboxylic acid group, which may be derivatised at a later stage in the method. This group may also be deprotonated to give an ionic backbone in the polymeric material.

In the method according to this invention the side chain precursors may have hydroxyl groups at each of their termini and each terminus reacts with a unit derived from maleic anhydride in different backbones to form a cross-linked polymeric material.

After reaction of the side chain precursors with the copolymer or terpolymer starting material, any unreacted units derived from maleic anhydride in the backbone may be ring-opened. This may be performed by hydrolysis, or using a base. The resulting product may be ionisable. This further reaction step has particular utility when there is a large proportion of maleic anhydride in the backbone, for instance in an alternating copolymer.

### Chewing Gum Composition

The chewing gum composition comprises a gum base, one or more sweetening or flavouring agents and a biologically active ingredient. Typically, the chewing gum composition comprises both a sweetening and a flavouring agent. The chewing gum composition may additionally comprise other agents, including nutraceutical actives, herbal extracts, stimulants, fragrances, sensates to provide cooling, warming or tingling actions, microencapsulates, abrasives, whitening agents and colouring agents.

The amount of gum base in the final chewing gum composition is typically in the range 5-95% by weight of the final composition, with preferred amounts being in the range 10-50% by weight, more preferably 15-25% by weight.

### Biologically Active Ingredient

The biologically active ingredient is any substance which modifies a chemical or physical process in the human or animal body. Preferably, it is a pharmaceutically active ingredient and is, for instance, selected from anti-platelet aggregation drugs, erectile dysfunction drugs, decongestants, anaesthetics, oral contraceptives, cancer chemotherapeutics, psychotherapeutic agents, cardiovascular agents, NSAID's, NO Donors for angina, non-opioid analgesics, antibacterial drugs, antacids, diuretics, anti-emetics, antihistamines, anti-inflammatories, antitussives, anti-diabetic agents (for instance, insulin), opioids, hormones and combinations thereof. Preferably, the active ingredient is a stimulant such as caffeine or nicotine. Alternatively, the active ingredient is an analgesic. A further example of an active ingredient is insulin.

In one embodiment of the invention, the biologically active ingredient is a non-steroidal anti-inflammatory drug (NSAID), such as diclofenac, ketoprofen, ibuprofen or aspirin. Alternatively the active ingredient is paracetamol (which is generally not classed as an NSAID).

In a different embodiment of the invention, the biologically active ingredient is a vitamin, mineral, or other nutritional supplement.

The biologically active ingredient may be an anti-emetic, for instance Dolasetron. Alternatively the biologically active ingredient is an erectile dysfunction drug, such as sildenafil citrate.

Generally the chewing gum composition comprises 0.01-20% wt active ingredient, more typically 0.1-5 wt %. The chewing gum composition may be in unit dosage form suitable for oral administration. The unit dosage form preferably has a mass in the range 0.5-4.5 g, for instance around 1 g. Generally, the chewing gum composition comprises 1-400 mg biologically active ingredient, more typically 1-10 mg, depending on the active ingredient. When the active ingredient is nicotine, for instance, the chewing gum composition typically comprises 1-5 mg nicotine. When the active ingredient is a non-steroidal anti-inflammatory drug, such as ibuprofen, the composition typically comprises 10-100 mg active ingredient.

Generally, the chewing gum composition will be chewed for up to an hour, although up to 30 minutes is more common. Preferably, after 30 minutes of chewing, at least 40%, more preferably at least 45%, most preferably at least 50% of the active ingredient present in the chewing gum composition has been released into the mouth. Depending on the nature of the active ingredient and its intended use, release may occur over a relatively longer or shorter period. For some active ingredients, for instance, a slow, sustained release is preferred, since this may reduce the active's side effects. This is the case for sildenafil citrate, as described in U.S. Pat. No. 6,592,850. In such cases, it is preferred that no more than 50% of the active is released after 15 minutes of chewing, and that active release still continues between 15 and 30 minutes after the commencement of chewing.

Alternatively, a faster rate of release may be preferable. Smokers using nicotine-replacement therapy, for instance, would prefer a faster delivery of nicotine to satisfy their nicotine craving. In such cases, it is preferred that 25-100% of the active is released after 10 minutes of chewing. More typically 35-65% of the active is released after 10 minutes of chewing. A fast release chewing gum composition that delivers a high total release of nicotine after a reasonable chewing time, has the advantage that less gum (i.e. less pieces of gum, or pieces with a lower mass) need to be purchased and chewed by the consumer. Alternatively, and to the advantage of the manufacturer, less of the active needs to be added to the chewing gum composition.

The sweetening agent may be selected from a wide range of materials including water-soluble artificial sweeteners, water-soluble agents and dipeptide based sweeteners, including mixtures thereof. Preferably, the sweetening agent is sorbitol. The flavouring agents may be selected from synthetic flavouring liquids and/or oils derived from plants, leaves, flowers, fruits (etc.), and combinations thereof. Suitable sweetening and flavouring agents are described further in U.S. Pat. No. 4,518,615.

The chewing gum composition of the present invention may comprise additional amphiphilic polymeric material (i.e. additional to the polymeric material that may be present in the chewing gum base), in addition to the chewing gum base, sweetening agent and flavouring agent. Preferably, this additional polymeric material, if present, comprises 1-20%, more preferably 3-15% by weight of the chewing gum composition. It may be soluble or insoluble in water.

### Method of Forming Chewing Gum Composition

The method typically comprises forming a chewing gum composition by blending the gum base with the biologically active ingredient and sweetening and flavouring agents. Standard methods of production of chewing gum compositions are described in Formulation and Production of Chewing and Bubble Gum. ISBN: 0-904725-10-3, which includes manufacture of gums with coatings and with liquid centres.

Typically, chewing gum compositions are made by blending gum base with sweetening and flavouring agents in molten form, followed by cooling of the blend. Such a method may be used in the present invention.

The inventors have found that controlled conditions of temperature facilitate the incorporation of biologically active ingredient into a chewing gum composition.

In the laboratory, a HAAKE MiniLab Micro Compounder (Thermo Fisher Corporation) may be used to form both the gum base and the chewing gum composition.

In the case of the gum base, the ingredients are typically mixed together by adding them in stages at a temperature in the range 80-120° C., typically around 100° C. After the gum base has formed, the material is extruded out of the MiniLab.

It will be noted that the MiniLab Compounder would not be used to mix large scale batches of chewing gum. An industrial scale machine, such as a Z-blade mixer would be used in this case.

The chewing gum composition may require heating to a temperature of around 100° C. (for instance, in the range 80-120° C.) in order to uniformly mix the components. This may present a problem when the biologically active ingredient is temperature sensitive, i.e. is unstable at such high temperatures. If the active ingredient is temperature sensitive, it is preferred that step (ii) of the method is carried out in two distinct stages. The first stage should be a mixing step wherein the chewing gum base is mixed with one or more sweetening and/or flavouring agents, and heated. This mixture is then cooled to a temperature at which the active ingredient is stable, and the active ingredient is added to the cooled mixture, optionally together with one or more further sweetening and flavouring agents to form a chewing gum composition. Amphiphilic polymeric material as defined above in the first aspect of the invention is added at either the gum base-forming step, or in step (ii) when the chewing gum composition is formed. Polymeric material may be added during both of these steps.

Preferably the mixture is heated to a temperature in the range 80-120° C., typically around 100° C. The mixture is generally cooled to a temperature in the range 40-80° C., preferably 50-70° C.

After the mixing is complete, the chewing gum composition may be extruded.

The biologically active ingredient may be added in solid, molten or liquid form. Nicotine is generally added as an oil, for instance, although use of a solid form (e.g. nicotine on an ion exchange resin, such as Polacrilex™) is preferred. Before adding the active ingredient in step (ii) the active ingredient may be pre-mixed with polymeric material and/or sweetening agent. Preferably, the sweetening agent is sorbitol.

During any of the steps of the method, the mixture may be stirred to improve homogeneity.

Step (ii) may comprise use of compression to form the chewing gum composition.

A unit dosage form of the chewing gum composition may be formed by extruding the chewing gum and shaping the extrudate to the desired form. The unit dosage form typically has a mass in the range 0.5-2.5 g, typically around 1 g. The dosage unit may take the form of a cylindrical or spherical body, or a tab.

Typically, the chewing gum composition comprises 5-95% by weight, preferably 10-50% by weight, more preferably 15-45% of the chewing gum base. Additional polymeric material may also be added to form the chewing gum composition, in an amount such that it comprises 1-15%, more preferably 3-15% of the chewing gum composition.

The steps to form the chewing gum composition may be carried out sequentially in the same apparatus, or may be carried out in different locations, in which case there may be intermittent cooling and heating steps.

In this method, the chewing gum base may have any of the preferred features discussed above.

One embodiment of this invention provides an amphiphilic polymeric material which has a straight or branched chain carbon-carbon backbone, and a multiplicity of side chains attached to the backbone, for use in the delivery of biologically active ingredient to a human or animal body.

The amphiphilic material and biologically active ingredient are as described above for the other aspects of this invention.

Delivery may be orally, intravenously, rectally, parenterally, by inhalation, topically, ocularly, nasally or to the buccal cavity.

The amphiphilic polymeric material may be formulated together with the biologically active material into the form of a composition suitable for the intended delivery. The compositions may be formulated in a manner known to those skilled in the art so as to give a controlled release, for example rapid release or sustained release, of the compounds of the present invention. Preferably, the composition is a pharmaceutical composition.

Pharmaceutically acceptable carriers suitable for use in such compositions are well known in the art. The compositions of the invention may contain 0.1-99% by weight of biologically active compound. The compositions of the invention are generally prepared in unit dosage form. Preferably, a unit dose comprises the active compound in an amount of 1-500 mg. The excipients used in the preparation of these compositions are the excipients known in the art. Compositions for oral administration include known pharmaceutical forms for such administration, for example tablets, troches, lozenges, aqueous or oily suspensions, dispersible powders or granules, emulsions, hard or soft capsules, or syrups and elixirs. The composition may also be a chewing gum, as detailed for the first aspect of this invention. The compositions may contain one or more agents such as sweetening agents, flavouring agents, colouring agents and preserving agents, in order to provide pharmaceutically elegant and palatable preparations.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

The composition may alternatively be in the form of an aqueous or oily suspension.

The pharmaceutical compositions may be in the form of a sterile injectable aqueous or oleagenous suspension. This suspension may be formulated according to the known art using those suitable dispersing or wetting agents and suspending agents.

Compositions for topical administration may also be suitable for use in the present invention. The active compound may be dispersed in a pharmaceutically acceptable cream, ointment or gel.

The invention will now be illustrated further in the following Examples, and with reference to the accompanying drawings, in which:

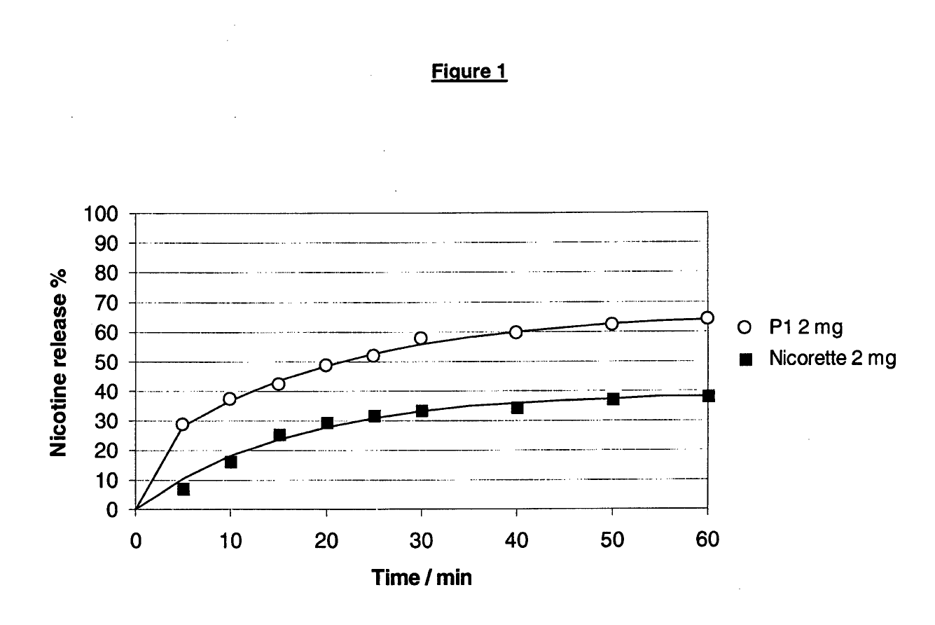

FIG. 1 shows accumulative nicotine release from commercial and P1 containing gums as determined using HPLC;

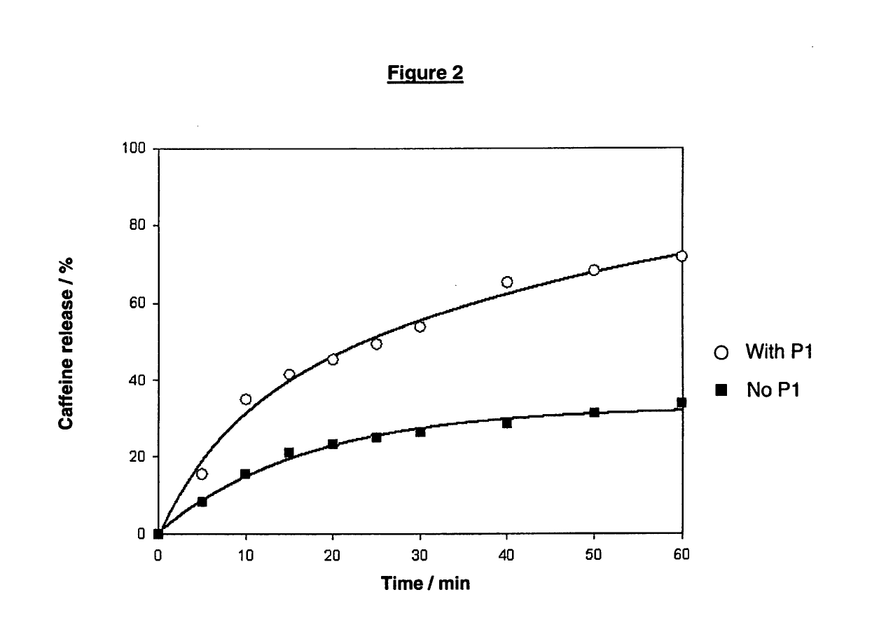

FIG. 2 shows accumulative caffeine release in artificial saliva as determined using HPLC from a gum sample containing P1, compared with a control gum sample not containing P1;

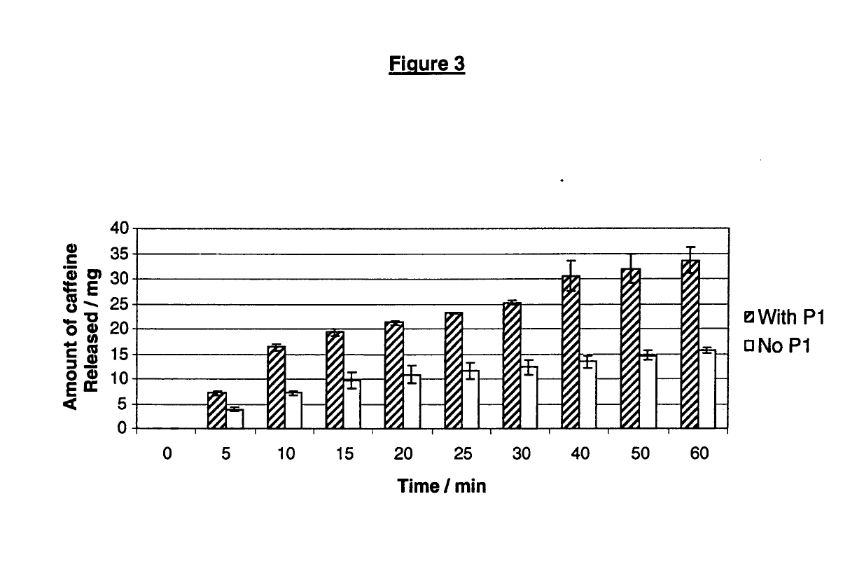

FIG. 3 shows caffeine release over time from the two samples in FIG. 2;

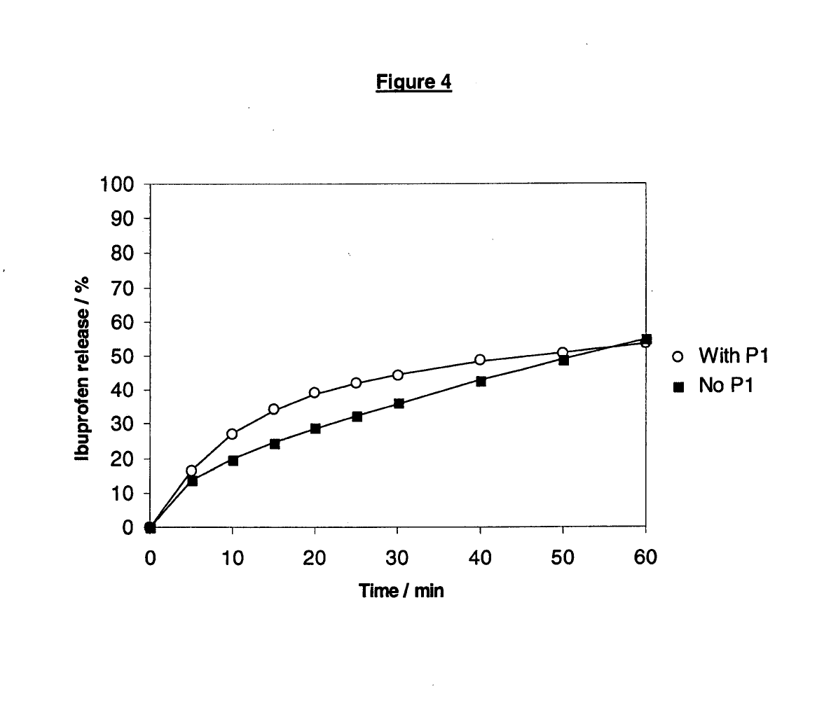

FIG. 4 compares accumulative Ibuprofen release in artificial saliva from gum containing P1, and a control gum as determined by using HPLC;

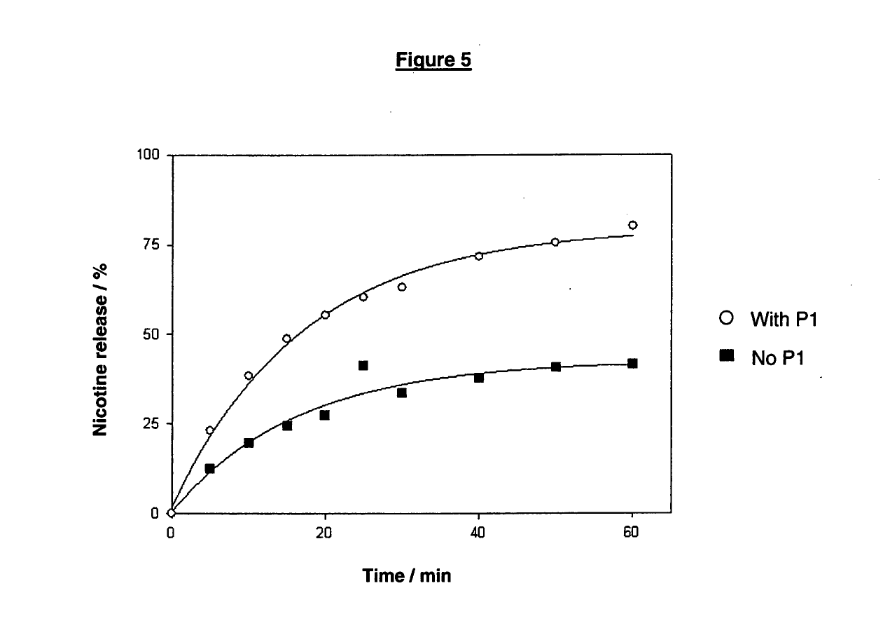

FIG. 5 compares accumulative nicotine release from gum containing P1, and a control gum, both made using nicotine polacrilex as determined using HPLC;

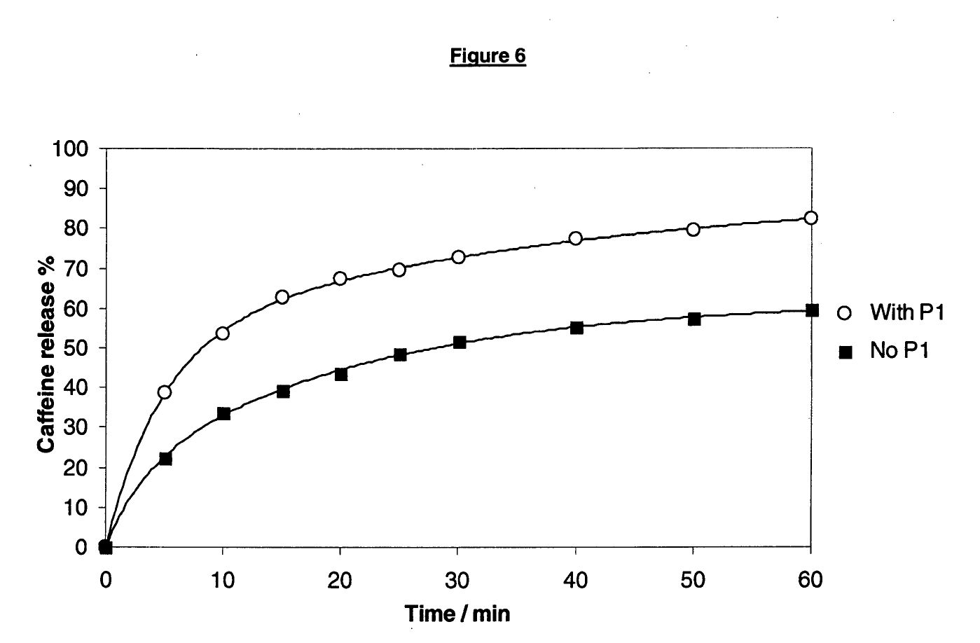

FIG. 6 compares accumulative caffeine release in artificial saliva from gum containing P1, and a control gum determined using HPLC;

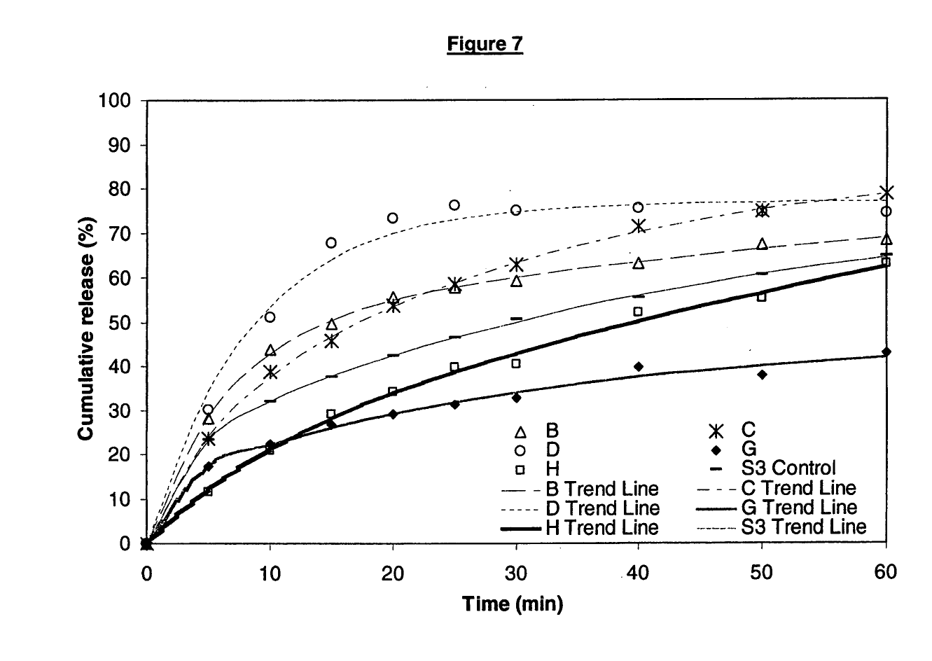

FIG. 7 shows cinnamaldehyde release from chewing gums; and

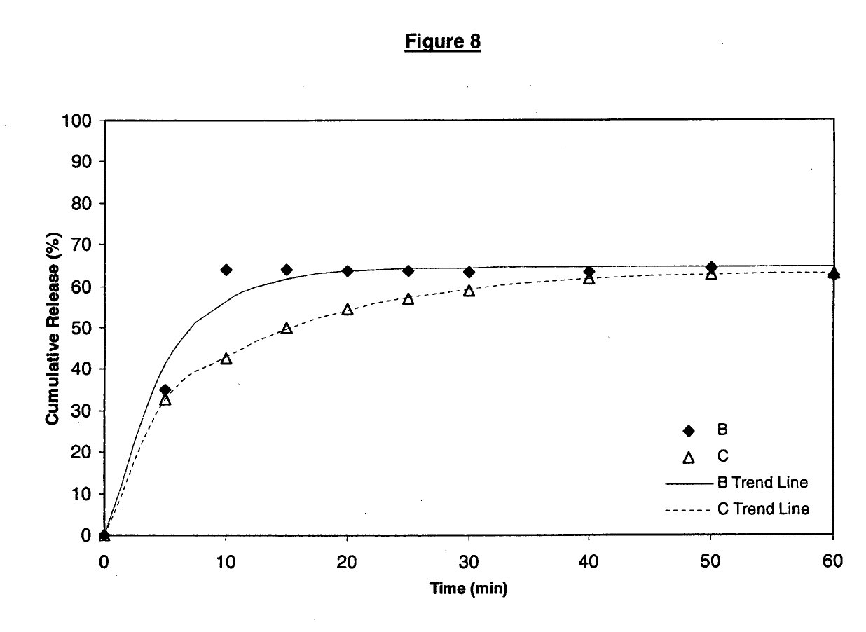

FIG. 8 shows the release of Ibuprofen from samples.

### REFERENCE EXAMPLE A

### Reaction of polyisoprene-graft-maleic Anhydride with poly(ethylene glycol) methyl ether (Preparation of P1)

PIP-g-MA (3.50 Kg, polyisoprene-graft-maleic anhydride obtained from Kuraray, LIR-403 grade) having the CAS No. 139948-75-7, an average M_{w} of approximately 25,000 and a typical level of grafting of MA of around 1.0 mol %, and poly(ethylene glycol) methyl ether (PEGME) (2.67 kg, purchased from Aldrich), having an average molecular weight of 2000 were weighed out and added to an air-tight jacketed reactor with a twenty litre capacity, equipped with an overhead stirrer. Toluene (8.15 Kg) was added to the reactor to dissolve the starting materials, and a flow of nitrogen gas passed through the vessel.

The vessel was then heated to reflux the toluene (115-116° C.) using an oil bath set to 140° C. connected to the reactors jacket. A Dean-Stark trap and condenser between the vessel and nitrogen outlet were used in order to remove any water from the poly(ethylene glycol) methyl ether and toluene by means of azeotropic distillation. Thus water was collected in the Dean-Stark trap over the course of the reaction.

The reaction mixture was refluxed for a total of approximately 37.5 hours. The reaction can also be catalysed by addition of acid or base. The product was purified in 2 L batches by adding the still warm (50° C.) material to 3 L tanks of deionised water. In the case of each batch the water was removed by filtration and the process of washing the graft copolymer with deionised water, and removing the water wash with the aid of filtration repeated a further five times. The product was dried under vacuum at 50° C. for 1 week.

The ¹H NMR spectrum was obtained using a Delta/GX 40 NMR spectrometer, operating at 400 MHz, in CDCl₃ (deuterated chloroform). P1 was obtained.

### REFERENCE EXAMPLE B

### Reaction of polyisoprene-graft-monoacid monomethyl ester with poly(ethylene glycol) methyl ether (Preparation of P2)

Poly(ethylene glycol) methyl ether was reacted with PIP-g-MaMme (polyisoprene-graft-monoacid monomethyl ester supplied by Kuraray Co. Ltd., LIR-410 grade). This PIP-g-MaMme has a functionality of 10 (i.e. carboxylic acid groups per molecule), and a molecular weight of approximately 25,000.

Poly(ethylene glycol) methyl ether (PEGME) (2.60 kg, purchased from Aldrich), having an average molecular weight of 2000 was weighed out and added to an air-tight jacketed reactor with a twenty litre capacity, equipped with an overhead stirrer. The PEGME was melted by heating it to 60° C. and PIP-g-MaMme (3.20 Kg) followed by toluene (7.35 Kg) were added into the reactor, and a flow of nitrogen gas passed through the vessel whilst the materials were mixed.

The vessel was then heated to reflux the toluene (115-116° C.) using an oil bath set to 140° C. connected to the reactor's jacket. A Dean-Stark trap and condenser between the vessel and nitrogen outlet were used in order to remove any water from the poly(ethylene glycol) methyl ether and toluene by means of azeotropic distillation. Thus water was collected in the Dean-Stark trap over the course of the reaction.

The reaction mixture was refluxed for a total of approximately 98.5 hours. The reaction can also be catalysed by addition of acid or base. The product was purified in 2 L batches typically by adding the still warm (50° C.) material to 3 L tanks of deionised water. In the case of each batch the water was removed by filtration and the process of washing the graft copolymer with deionised water, and removing the water wash with the aid of filtration repeated a further five times. The product was dried under vacuum at 50° C. for 1 week.

The ¹H NMR spectrum was obtained using a Delta/GX 40 NMR spectrometer, operating at 400 MHz, in CDCl₃ (deuterated chloroform). P2 was obtained.

The physical properties of the polymers are given in Table 2.

TABLE 2
Polymer Physical Properties:

| Name | Backbone/Side Chain | Mₙ | Tₘ | Quantity |
|---|---|---|---|---|
| P1 | PIP-g-MA/PEO 2K | 30K | 48° C. | 4967 g |
| P2 | PIP-g-MaMme/PEO 2K | 45K | 43° C. | 4896 g |

The values in the column headed Tₘ is the maxima of a transition believed to be associated with the melting of the PEG chains of the graft copolymers

### REFERENCE EXAMPLE C

### PEG Grafting of Polymers with Maleic Anhydride in their Backbones

### Maleic Anhydride Copolymers

### Poly(isobutylene-alt-maleic anhydride)

Two molecular weights (Mₙ: 6000, 60 000 g mol⁻¹, as declared by the supplier), both were obtained from the Sigma-Aldrich company.

### Poly(maleic anhydride-alt-1-octadecene)

Molecular weight 30-50 000 g mol⁻¹ (as declared by the supplier) obtained from the Sigma-Aldrich company.

### Ethylene-Maleic Anhydride Terpolymers

These are random copolymers of ethylene, maleic anhydride, and another monomer.

### Poly(ethylene-co-butyl acrylate-co-maleic anhydride)

This is a copolymer of ethylene (91 weight percent), N-butyl acrylate (6%), and maleic anhydride (3%). This material was obtained from Sigma-Aldrich (molecular weight undisclosed and propriety information).

### Poly(ethylene-co-vinyl acetate-co-maleic anhydride)

This is a copolymer of ethylene, vinyl acetate and maleic anhydride. The polymer was obtained from Arkema and sold under the Orevac trade name (grade 9304 was used).

### Side Chains Precursors

In all cases the graft was methoxy poly(ethylene glycol) (MPEG), also known as poly(ethylene glycol) methyl ether (PEGME). Material was obtained from two suppliers, the Sigma-Aldrich company, and Clariant (sold as Polyglykol M 2000S). In both cases the polymers were sold as having a molecular weight of 2000, and are believed to be have a very similar chemical structure and properties. Polymers A, C-E and G (Table 3) were synthesised using the Aldrich material, the others using the Clariant material.

### Graft Copolymers

By “graft copolymer”, we mean “polymeric material”, and these two terms are used interchangeably.

A number of graft copolymers where synthesised by grafting MPEG to the backbones described above.

TABLE 3
Polymers Examined.

| | | | | | Backbone | |
|---|---|---|---|---|---|---|
| | | | | | MA | MA |
| Polymer | | Backbone | | Graft | Loading | Targeted |
| Sample | Backbone | Mₙ | Graft | Mₙ | (weight %) | (mol %)^{c} |
| A | P(IB-alt-MA) | 6000 | MPEG | 2000 | 64^{a} | 10 |
| B | P(IB-alt-MA) | 6000 | MPEG | 2000 | 64^{a} | 28 |
| C | P(IB-alt-MA) | 60 000 | MPEG | 2000 | 64^{a} | 10 |
| D | P(MA-alt-O) | 30-50 000 | MPEG | 2000 | 28^{a} | 11 |
| E | P(MA-alt-O) | 30-50 000 | MPEG | 2000 | 28^{a} | 11 |
| F | P(MA-alt-O) | 30-50 000 | MPEG | 2000 | 28^{a} | 100 |
| G | P(E-co-BA-co-MA) | Not known | MPEG | 2000 | 3 | 100 |
| H | P(E-co-VA-co-MA) | Not known | MPEG | 2000 | 3^{b} | 50 |
| I | P(E-co-VA-co-MA) | Not known | MPEG | 2000 | 3^{b} | 100 |

^{a}= Polymers are approximately 50 mol % MA, value for weight % depends on Fw of monomer,
^{b}= Backbone loading variable between 1.6-3.2%, values calculated using 3.2%,
^{c}= percentage of available MA targeted for reaction.

As will be apparent from Table 3, often not all of the MA was targeted for reaction. For instance, in the case of Polymer samples A-E only a proportion of the maleic anhydride in the alternating copolymer backbone reacted. This leaves a number of maleic anhydride rings present on the backbones which can themselves be exploited by ring opening (see section on emulsification). It may be noted that in some cases not all of the maleic anhydride targeted for reaction with MPEG may have been reacted.

### Synthesis of the Graft Copolymer

### Polymer A:

Poly(isobutylene-aft-maleic anhydride) (Mₙ: 6000 g mol⁻¹, 40 g) and polyethylene glycol) methyl ether (Mₙ: 2000 g mol⁻¹, 50 g) were dissolved in a mixture of DMF (100 mL) and toluene (100 mL) in a reaction flask. The flask was heated at reflux temperature under nitrogen gas for 24 h, any water present being removed from the reaction by means of azeotropic distillation and collection into a Dean-Stark apparatus. The resulting polymer solution was cooled and precipitated into diethyl ether, the polymer recovered using filtration, and dried to remove traces of solvent. The grafting of MPEG onto the backbone was confirmed using infra-red spectroscopy using a Bruker spectrometer by observing changes in the region 1700-1850 cm⁻¹ associated with the maleic anhydride units.

### Polymer B:

Polymer B was synthesized in the same manner as Polymer A using poly(ethylene glycol) methyl ether (Mₙ: 2000 g mol⁻¹, 110 g) as the graft. Reaction was allowed to continue for a total of 36 h. The polymer was characterised in a similar manner to polymer A.

### Polymer C:

Polymer C was synthesized in the same manner as Polymer A using Poly(isobutylene-a/t-maleic anhydride) (Mₙ: 60 000 g mol⁻¹, 40 g) as the backbone. The polymer was characterised in a similar manner to polymer A.

### Polymer D:

Polymer D was synthesized in the same manner as Polymer A using poly(maleic anhydride-alt-1-octadecene) (Mₙ: 30-50 000 g mol⁻¹, 50 g) as the backbone and poly(ethylene glycol) methyl ether (Mₙ: 2000 g mol⁻¹, 30 g) as the graft. Toluene (200 mL) was used as the reaction solvent; in this case the polymer solution was precipitated in water. The amphiphilic nature of the resulting graft copolymer led to a poor yield (25% of the theoretical). The polymer was characterised in a similar manner to polymer A.

### Polymer E:

Polymer E was synthesised in the same manner as Polymer D except that the polymer solution was not precipitated in water, instead the reaction solvent was removed under vacuum. This material was consequently isolated in a higher yield than D, and may be suitable for applications where excess PEG in the final product is not a critical issue. The polymer was characterised in a similar manner to polymer A.

### Polymer F:

Polymer F was synthesised in the same manner as Polymer D using poly(maleic anhydride-alt-1-octadecene) (Mₙ: 30-50 000 g mol⁻¹, 20 g) poly(ethylene glycol) methyl ether (Mₙ: 2000 g mol⁻¹, 136 g) as the graft. Toluene (500 mL) was used as the reaction solvent; the polymer solution was precipitated in hexane. Reaction was allowed to continue for a total of 36 h. The polymer was characterised in a similar manner to polymer A. Excess PEG may be removed from the polymer via dialysis or a similar methodology.

### Polymer G:

Polymer G was synthesized in the same manner as Polymer A using poly(ethylene-co-butyl acrylate-co-maleic anhydride) (40 g) as the backbone and poly(ethylene glycol) methyl ether (Mₙ: 2000 g mol⁻¹, 30 g) as the graft. A mixture of xylene (100 mL) and toluene (100 mL) was used as the reaction solvent; in this case the polymer solution was precipitated in ethanol. The polymer was characterised in a similar manner to polymer A.

### Polymer H:

Polymer H was synthesized in the same manner as Polymer A using poly(ethylene-co-vinyl acetate-co-maleic anhydride) (40 g) as the backbone and poly(ethylene glycol) methyl ether (Mₙ: 2000 g mol⁻¹, 13 g) as the graft. A mixture of xylene (125 mL) and toluene (125 mL) was used as the reaction solvent; in this case the polymer solution was precipitated in ethanol. The polymer was characterised in a similar manner to polymer A.

### Polymer I:

Polymer I was synthesized in the same manner as Polymer H using poly(ethylene glycol) methyl ether (Mₙ: 2000 g mol⁻¹, 39 g) as the graft. The polymer was washed thoroughly with more ethanol after filtration to remove PEG from the polymer. The polymer was characterised in a similar manner to polymer A.

### REFERENCE EXAMPLE D

### Drug Release Tests on Medicated Chewing Gums—Experimental Method

Each pre-shaped piece of gum was weighed before chewing, and the weight recorded to allow estimation of the total quantity of drug in each piece.

A ‘ERWEKA DRT-1’ chewing apparatus from AB FIA was used, which operates by alternately compressing and twisting the gum in between two mesh grids. A water jacket, with the water temperature set to 37° C. was used to regulate the temperature in the mastication cell to that expected when chewed in vivo, and the chew rate was set to 40 ‘chews’ per minute. The jaw gap was set to 1.6 mm.

40 mL artificial saliva (composed of an aqueous solution of various salts, at approx pH 6—see below, Table 4) was added to the mastication cell, then a plastic mesh placed at its bottom. A piece of gum of known weight was placed on the centre of the mesh, and a second piece of mesh put on top.

### Artificial Saliva:

TABLE 4
Artificial Saliva Formulation

| Components | Quantity (mmol/L) |
|---|---|
| KH₂PO₄ | 2.5 |
| Na₂HPO₄ | 2.4 |
| KHCO₃ | 15 |
| NaCl | 10 |
| MgCl₂ | 1.5 |
| CaCl₂ | 1.5 |
| Citric acid | 0.15 |

PH adjusted to 6.7 with HCl

Procedure for Analysing the Release Profiles of Active Ingredients from Gum

The parameters in Table 5 were always used in chewing unless otherwise noted.

TABLE 5
Chewing Parameters

| Parameter | Value |
|---|---|
| Temperature | 37° C. |
| Gaps between jaws | 1.6 mm |
| Twisting angle | 20° |
| Chew Frequency | 40 strokes/min |

At the start of each run, the cell containing the artificial saliva and gum was left for 5 minutes so that the system could equilibrate to 37° C. The gum was then masticated. A sample volume of 0.5 mL was then withdrawn from the test cell periodically during a release run (5, 10, 15, 20, 25, 30, 40, 50 and 60 minutes).

All the samples were then analysed by HPLC using a typical Perkin Elmer HPLC Series 200 system, equipped with an autosampler, pump, and diode array detector. Data handling and instrument control was provided via Totalchrom v 6.2 software. The columns and mobile phase were adjusted to the active ingredient as follows:

Ibuprofen HPLC details: Column: Hypersil C18 BDS, 150×4.6 mm; Mobile phase: Acetonitrile/0.05% aqueous orthophosphoric acid in a 60/40 ratio, 1 mL/min; UV detector, wavelength—220 nm.

Caffeine HPLC details: Column—Polaris C18-A 58, 250×4.6 mm. Mobile phase—Acetonitrile/0.05% aqueous orthophosphoric acid in a 60/40 ratio. Flow rate—1 mL/min UV detector wavelength—270 nm

Nicotine HPLC details: Column—Hypersil Gold C18 58, 250×4.6 mm. Mobile phase —Acetonitrile/0.05M aqueous ammonium dihydrogen phosphate pH 8.5 (pH adjusted with ammonium hydroxide) in a 30/70 ratio. Flow rate—1 mL/min .UV detector wavelength—260 nm.

Cinnamaldehyde details: Column—Varian Polaris 5u C18-A 250×4.6 m. Mobile Phase—Acetonitrile/0.05% orthophosphoric acid (60/40). Flow rate—1 mL/min. Detection—UV 250 nm. Inj vol—5 uL

Two injections into the HPLC column were used for each sample, to ensure reproducibility.

### Preparation of Gum Base and Chewing Gum

### Chemicals

Calcium carbonate (CaCO₃), ester gum, hydrogenated vegetable oil (HVO), polyisobutylene (PIB), polyvinyl acetate) (PVAc), glyceromonostearate (GMS), microwax, sorbitol liquid, sorbitol solid, and peppermint oil, were all food grade materials obtained from the Gum Base Company. Ibuprofen (40 grade) was obtained from Albemarle; ketoprofen, naproxen, and (−) nicotine oil were obtained from the Sigma-Aldrich company. Nicotine polacrilex was obtained from Siegfried.

### Mixing of the Chewing Gum and Chewing Gum Base

The gum base and chewing gum were mixed using a HAAKE MiniLab Micro Compounder (i.e. lab mixer) manufactured by the Thermo Fisher Corporation. The screws were set to co-rotate at 80 turns/min.

In the case of the gum base, the ingredients were typically mixed together by adding them in four different stages at 100° C. At each stage the ingredients were fed together into the HAAKE MiniLab, and mixed for a set period of time, prior to the next stage being carried out. After the final stage, the material was then extruded out of the MiniLab Compounder.

The chewing gum was also made on the MiniLab in a similar multi-step manner. A portion of the gumbase was typically fed back into the compounder in the first step with sorbitol, and mixed at 100° C. during the first stage. The mixer was allowed to cool to 60° C., and the active/P1 mix added with appropriate ingredients. If the active ingredient and other additives are stable at 100° C., it may be preferable to mix the ingredients at this temperature. After the mixing was complete, the gum was extruded from the MiniLab compounder.

### EXAMPLE 1

### Formulation of Gum Containing Nicotine

### Objective

To blend a chewing gum containing 2 mg nicotine, with the nicotine pre-blended with polymeric material in chloroform.

### Experimental

3 g P1 (prepared as in Reference Example A) was dissolved in 5 mL chloroform, then 0.1 mL nicotine oil was added. The mixture was stirred thoroughly, then poured into a Petri dish and allowed to dry in a fume hood overnight. It was left for a further 3 hours in a vacuum oven at room temperature to remove all traces of chloroform. The nicotine/P1 mixture was then blended into R3 gum base in the sweetener stage using the formulation, as described in Table 6 below. In this table, each stage refers to a particular step wherein the compounds in the “chemicals” section are mixed.

TABLE 6
Formulation details for R3 gum base

| Stage | Chemicals | Quantities | Temperature | Duration |
|---|---|---|---|---|
| 1 | PIB | 13% = 1.04 g | 100° C. | 10 min |
| | PVAc | 6% = 0.48 g | | |
| | CaCO₃ | 6% = 0.48 g | | |
| | Ester gum | 3.6% = 0.288 g | | |
| 2 | CaCO₃ | 9% = 0.72 g | 100° C. | 20 min |
| | Ester gum | 5.4% = 0.432 g | | |
| 3 | PVAc | 9% = 0.72 g | 100° C. | 20 min |
| | CaCO₃ | 15% = 1.2 g | | |
| | Ester gum | 9% = 0.72 g | | |
| 4 | Glyceromonostearate | 6% = 0.48 g | 100° C. | 20 min |
| | HVO | 12% = 0.96 g | | |
| | P1 | 6% = 0.48 g | | |

TABLE 7
Formulation details for finished gum

| | Chemicals | Quantities | Temperature | | Duration |
|---|---|---|---|---|---|
| 1 | Gum base | 39.0% = 3.12 g | 100° | C. | 30 min |
| | Sorbitol (s) | 22.1% = 1.77 g | | | |
| 2 | Cool to 60° C. whilst | | 100-60° | C. | 30 min |
| | mixing | | | | |
| 3 | P1 | 6.0% = 0.48 g | 60° | C. | 30 min |
| | Nicotine | 0.2% = 0.016 g | | | |
| | Sorbitol (l) | 9.5% = 0.76 g | | | |
| | Sorbitol (s) | 22.1% = 1.77 g | | | |
| | Peppermint flavour | 1% = 0.08 g | | | |

The gum was extruded as a homogeneous white tape, which was then shaped to form cylinders of gum by rolling between two glass surfaces to form a cylindrical shape.

### Results

HPLC was carried out for drug release analysis. Gum comprising 2 mg nicotine was “chewed” with 40 mL saliva (see Reference Example C, “Experimental” for details of the method used). The theoretical maximum release, assuming that all of the nicotine was released in the 40 mL of artificial saliva was 50 μg/mL. Nicorette™ gum was compared with gum comprising P1. Samples were taken every 5 minutes for HPLC analysis. FIG. 1 compares Nicotine release from Nicorette™ and P1 gum. Nicotine release is observed to be at a faster rate from the gum containing P1. As a result, the total release of nicotine from the gum containing P1 is observed to be higher after 5 min, and during the remainder of the course of the experiment.

### EXAMPLE 2

### Formulation of Control Gum Containing Nicotine

### Objective

To blend a chewing gum containing 2 mg nicotine but no polymeric material, with the drug pre-blended with microwax instead of polymeric material, in order to compare the effect of polymeric material versus normal gum on nicotine release.

### Experimental

3 g microwax was dissolved in 5 mL chloroform. To dissolve the wax it was necessary to leave the solution overnight on a ‘spinner’ to agitate the solution. After this time, the majority of the microwax had become dispersed in the chloroform, so 0.1 mL nicotine was added. The mixture was stirred thoroughly, then poured into a Petri dish and allowed to dry in a fume hood overnight. It was left for a further 3 hours in a vacuum oven at room temperature to remove all traces of chloroform. The nicotine/microwax mixture was then blended into S3 gum base in the sweetener stage using the low temperature formulation, as described below.

TABLE 8
Formulation details for S3 gum base (R3 with microwax rather than
polymeric material)

| Stage | Chemicals | Quantities | Temperature | Duration |
|---|---|---|---|---|
| 1 | PIB | 13% = 1.04 g | 100° C. | 10 min |
| | PVAc | 6% = 0.48 g | | |
| | CaCO₃ | 6% = 0.48 g | | |
| | Ester gum | 3.6% = 0.288 g | | |
| 2 | CaCO₃ | 9% = 0.72 g | 100° C. | 20 min |
| | Ester gum | 5.4% = 0.432 g | | |
| 3 | PVAc | 9% = 0.72 g | 100° C. | 20 min |
| | CaCO₃ | 15% = 1.2 g | | |
| | Ester gum | 9% = 0.72 g | | |
| 4 | Glyceromonostearate | 6% = 0.48 g | 100° C. | 20 min |
| | HVO | 12% = 0.96 g | | |
| | Microwax | 6% = 0.48 g | | |

TABLE 9
Formulation details for finished gum at 60° C.

| Stage | Chemicals | Quantities | Temperature | | Duration |
|---|---|---|---|---|---|
| 1 | Gum base | 39.0% = 3.12 g | 100° | C. | 30 min |
| | Sorbitol (s) | 22.1% = 1.77 g | | | |
| 2 | Cool to 60° C. | | 100-60° | C. | 30 min |
| | whilst mixing | | | | |
| 3 | Microwax | 6.0% = 0.48 g | 60° | C. | 30 min |
| | Nicotine | 0.2% = 0.016 g | | | |
| | Sorbitol (l) | 9.5% = 0.76 g | | | |
| | Sorbitol (s) | 22.1% = 1.77 g | | | |
| | Peppermint flavour | 1% = 0.08 g | | | |

The gum extruded as a homogeneous white tape, which was then shaped to form 1 g cylinders of gum. The gum was rolled between 2 glass surfaces to form a cylindrical shape.

### EXAMPLE 3

### Caffeine

Samples according to Table 10 were prepared using similar methods to those described in Examples 1 and 2, but using caffeine rather than nicotine.

The gum base was standard gum base R3, as described in Table 5.

TABLE 10

| Drug | Drug level | Any Polymeric Material? | Flavour |
|---|---|---|---|
| Caffeine | 4.7% w/w | no | mint |
| Caffeine | 4.7% w/w | yes, 10% P1 in gum base | mint |
| | | (4% P1 in final gum) | |

FIGS. 2 and 3 show that the presently described polymeric materials enhance caffeine release from gum samples; release from the sample containing P1 being faster, and greater, than that from the control without P1.

### EXAMPLE 4

### Formulation of Gum Base Containing Various NSAID Drugs

### Objective

To blend gum bases containing ibuprofen, ketoprof en and naproxen.

### Experimental

The drugs were first blended into a mix of HVO and amphiphilic polymeric material (P1) using the chewing-gum mixer. Some changes to the formulation method used in previous Examples were made.

The amphiphilic polymeric material was broken into small pieces before addition to the mixer, and the polymer and HVO were added alternately in small quantities to ensure an even distribution of the two.

The HVO/polymer mix was left for at least 10 minutes to mix before cooling. The mixture was cooled to 63° C. before addition of the drug, as this temperature is known to be a point at which most active ingredients, including ibuprofen, are effectively stable for at least the period of time required to manufacture the chewing gums described in this disclosure.

The drug was added gradually, over a period of 5-10 minutes to give an evenly spread distribution.

The HVO/polymer/drug mixture was mixed for at least 30 minutes for each gum; for some samples (e.g. 20 mg ibuprofen in P1 mix) the mixture still appeared fairly clear at this stage (through the view hole, without opening the mixer) suggesting not much drug had been mixed in, so the mixing was continued for up to an hour if necessary, until a white colour was observed.

TABLE 11
Drug solubilisation in HVO and P1

| Stage | Chemicals | Quantities | Temperature | Duration | |
|---|---|---|---|---|---|
| 1 | HVO | 10 g | 100° C. | 10 | min |
| | P1 polymer | 5 g | | | |
| 5 | Cool to 63° C. whilst | | 63° C. | 30 | min |
| | mixing | | | | |
| 4 | Ibuprofen | 0.56 g | 63° C. | 30-60 | min |

Gum was then extruded and ¹H NMR was performed to determine the amount of drug in the extrudates. This was done by comparing the ratios of the peaks resulting from the drug and those from the polyisoprene (in the P1) in the spectrum. Signals from aromatic groups in Ibuprofen, Ketoprofen, and Naproxen are typically observed at 7.1 ppm, 7.7 ppm, and 7.5 ppm respectively. In polyisoprene a peak is detected at around 5.1 ppm from the hydrogen atom adjacent to the double bond. This signal in polyisoprene results from one hydrogen atom per repeat unit in the polymer, and approximately 367 per polymer chain. An exemplary calculation, using the Ibuprofen mixture, is as follows:

Ratio of Ibuprofen: P1 signals=2:8.754, which corresponds to 1 mole Ibuprofen for each (8.754/367)=0.024 moles P1. Ibuprofen Fw=206 and P1 Mₙ=30,000. This gives a mass ratio of 1:3.47, giving 23.7 mg Ibuprofen per 1.5 g gum base portion. The results for all three drugs are shown in the table below.

TABLE 12
Extruded Gum Samples

| | | Target drug | Extruded drug | |
|---|---|---|---|---|
| | | quantity/gum | quantity/ | |
| Polymer | Drug | piece | gum piece | Comments |
| P1 | Ibuprofen | 20 mg | 23.7 mg | Homogeneous |
| P1 | Ketoprofen | 20 mg | 23.7 mg | Homogeneous |
| P1 | Naproxen | 20 mg | 21.7 mg | Homogeneous |

All drug/oil/polymer mixtures were then blended into gum base derived from the ‘R3’ formulation, as described below.

TABLE 13
Formulation details for R3 gum base with drug

| Stage | Chemicals | Quantities | Temperature | Duration |
|---|---|---|---|---|
| 1 | PIB | 13% = 1.04 g | 100° C. | 10 min |
| | PVAc | 6% = 0.48 g | | |
| | CaCO₃ | 6% = 0.48 g | | |
| | Ester gum | 3.6% = 0.288 g | | |
| 2 | CaCO₃ | 9% = 0.72 g | 100° C. | 20 min |
| | Ester gum | 5.4% = 0.432 g | | |
| 3 | PVAc | 9% = 0.72 g | 100° C. | 20 min |
| | CaCO₃ | 15% = 1.2 g | | |
| | Ester gum | 9% = 0.72 g | | |
| 4 | GMS | 6% = 0.48 g | 100° C. | 20 min |
| 5 | Cool to 60° C. | | 100-60° C. | 30 min |
| | whilst mixing | | | |
| 6 | HVO | 12% = 0.96 g | 60° C. | 20 min |
| | P1 | 6% = 0.48 g | | |
| | Drug | 0.67% = 0.11 g | | |

### EXAMPLE 5

### Formulation of Chewing Gum with Amphiphilic Polymeric Material and Ibuprofen Blended in Bulk

### Objective

To add the amphiphilic polymeric material and drug right at the end of the chewing gum formulation, pre-blended in the mixer (without solvent). This allowed the rest of the chewing gum to be blended at 100° C. as would be normal for gum. This may help to improve cohesion of the finished gum.

Mixing of P1 with Ibuprofen:

TABLE 14
Formulation details for P1 ibuprofen mix

| Stage | Chemicals | Quantities | Temperature | Duration |
|---|---|---|---|---|
| 1 | P1 | 75% = 6.0 g | 100° C. | 10 min |
| Cool to 60° C. whilst stirring | | | 100-60° C. | 30 min |
| 2 | Ibuprofen | 25% = 2.0 g | 60° C. | 20 min |

For details of the “R3” gum base used in this formulation, see Table 6.

TABLE 15
Formulation details for finished gum

| Stage | Chemicals | Quantities | Temperature | Duration |
|---|---|---|---|---|
| 1 | Gum base | 37.5% = 3.0 g | 100° C. | 15 min |
| | Sorbitol (l) | 10% = 0.8 g | | |
| | Sorbitol (s) | 6% = 1.36 g | | |
| 2 | Sorbitol (l) | 3% = 0.24 g | 100° C. | 15 min |
| | Sorbitol (s) | 25.5% = 2.04 g | | |
| | Peppermint flavour | 1% = 0.08 g | | |
| Cool to 60° C. whilst | | | 100-60° C. | 30 min |
| stirring | | | | |
| 3 | P1 | 6% = 0.48 g | 60° C. | 15 min |
| | Ibuprofen | 2% = 0.16 g | | |

This product extruded as a malleable, white solid. The fact that the majority of the gum was blended at 100° C. seemed to have improved characteristics of the gum, for instance, its flexibility.

### EXAMPLE 6

### Formulation of Chewing Gum with Ibuprofen Added at the End

### Objective

Formulating a gum where the final stage is carried out at 60° C. (to avoid risk of degradation of sensitive actives) is challenging, as a chewing gum with poor cohesion and chewing characteristics can result from the lower mixing temperature. This Example therefore aimed to study the possibility of adding the drug right at the end of the formulation, so that the rest of the blending can be carried out at 100° C. as normal. In this case 20 mg ibuprofen was dispersed in every 1 g piece of chewing gum.

For details of the “R3” gum base formulation used in this formulation, see Table 6.

TABLE 16
Formulation details for finished gum, with ibuprofen

| Stage | Chemicals | Quantities | Temperature | Duration |
|---|---|---|---|---|
| 1 | Gum base | 37.5% = 3.0 g | 100° C. | 15 min |
| | Sorbitol (l) | 10% = 0.8 g | | |
| | Sorbitol (s) | 6% = 1.36 g | | |
| 2 | Sorbitol (l) | 3% = 0.24 g | 100° C. | 15 min |
| | Sorbitol (s) | 25.5% = 2.04 g | | |
| | P1 | 6% = 0.48 g | | |
| | Peppermint | 1% = 0.08 g | | |
| | flavour | | | |

| | | |
|---|---|---|
| Cool to 60° C. whilst | 100-60° C. | 30 min |
| stirring | | |

| | | | |
|---|---|---|---|
| Ibuprofen | 0.8 g | 60° C. | 30 min |

The extruded white gum could be shaped successfully, but was not as malleable as those in Example 7.

### EXAMPLE 7

### Formulation of Chewing Gum with Polymeric Material and Ibuprofen Blended in Bulk and Added with Sorbitol −20 Mg

### Objective

To blend the polymeric material and drug into a different formulation at 60° C., with the aim of producing a gum with better cohesion than previous low-temperature blends.

### Experimental

The ibuprofen was pre-blended with polymeric material and added in the same stage as the sorbitol and flavouring.

Mixing of Polymeric Material with Ibuprofen:

TABLE 17
Formulation details for polymer/ibuprofen mix

| Stage | Chemicals | Quantities | Temperature | Duration |
|---|---|---|---|---|
| 1 | P1 | 75% = 6.0 g | 100° C. | 10 min |
| Cool to 60° C. whilst | | | 100-60° C. | 30 min |
| stirring | | | | |
| 2 | Ibuprofen | 25% = 1.96 g | 60° C. | 10 min |

This blend was carried out for both P1 and P2. The mixing was carried out for 10 min after which the product was inspected and if necessary it was blended for another 10 min.

The quantity of ibuprofen present in each blend was calculated from the ¹H NMR peak ratios, using the method shown in Example 4:
P1 sample: Peak ratio=2.0:7.73
   Equivalent to 26.8 mg ibuprofen per 1.0 g chewing gum
P2 sample: Peak ratio=2.0:4.80
   Equivalent to 28.9 mg ibuprofen per 1.0 g chewing gum

Both blends used the same R3 gum base formulated with P1 (Table 6).

The conditions for blending into fully formulated gum are shown in Table 18:

TABLE 18
Formulation details for finished gum

| Stage | Chemicals | Quantities | Temperature | Duration |
|---|---|---|---|---|
| 1 | Gum base | 37.5% = 3.06 g | 100° C. | 15 min |
| | Sorbitol (s) | 21.25% = 1.73 g | | |
| Cool to 60° C. | | | 100-60° C. | 30 min |
| whilst mixing | | | | |
| 2 | Sorbitol (l) | 9.1% = 0.74 g | 60° C. | 15 min |
| | Sorbitol (s) | 21.25% = 1.73 g | | |
| | P1/2 | 6% = 0.49 g | | |
| | Ibuprofen | 2% = 0.16 g | | |
| | Peppermint | 1% = 0.08 g | | |
| | flavour | | | |

Both products extruded as malleable white solids

See Example 9 for a comparison of the ibuprofen release from this gum and a control gum without P1, made with an otherwise comparative formulation and methodology.

### EXAMPLE 8

### Formulation of Control Samples: Ibuprofen Chewing Gum without Amphiphilic Polymeric Material

### Objective

To formulate chewing gums with ibuprofen incorporated in the final stage but which do not contain any polymeric material, as control samples for release experiments.

Formulation for 20 mg ibuprofen/g chewing gum, using S3 gum base (Table 8):

TABLE 19
Formulation details for finished gum

| Stage | Chemicals | Quantities | Temperature | Duration |
|---|---|---|---|---|
| 1 | Gum base | 37.5% = 3.0 g | 100° C. | 15 min |
| | Sorbitol (l) | 10% = 0.8 g | | |
| | Sorbitol (s) | 17% = 1.36 g | | |
| 2 | Sorbitol (l) | 3% = 0.24 g | 100° C. | 15 min |
| | Sorbitol (s) | 25.5% = 2.04 g | | |
| | Peppermint flavour | 1% = 0.08 g | | |
| Cool to 60° C. | | | 100-60° C. | 30 min |
| whilst mixing | | | | |
| 3 | Ibuprofen | 2% = 0.16 g | 60° C. | 30 min |

### EXAMPLE 9

### Formulation of Control Sample with Microwax and Ibuprofen Pre-Blended in Bulk and Added Last

### Objective

To blend a control sample of gum with microwax instead of P1, for comparison with Example 7.

### Experimental

The mixture was a repeat of Example 7 using microwax in place of P1. The quantities for this blend are scaled up slightly to allow for the reduction in sorbitol liquid used, to ensure the blend still reaches 8 g.

TABLE 20
Formulation details for microwax/ibuprofen mix

| Stage | Chemicals | Quantities | Temperature | Duration |
|---|---|---|---|---|
| 1 | Microwax | 75% = 6.0 g | 100° C. | 10 min |
| | Cool to 60° C. whilst stirring | | 100-60° C. | 30 min |
| 2 | Ibuprofen | 25% = 1.96 g | 60° C. | 15 min |

An “S3” gum base formulation was used as shown in Table 8.

This was then blended into fully formulated gum:

TABLE 21
Formulation details for finished gum at 60° C.

| Stage | Chemicals | Quantities | Temperature | Duration |
|---|---|---|---|---|
| 1 | Gum base | 37.5% = 3.06 g | 100° C. | 15 min |
| | Sorbitol (s) | 21.25% = 1.73 g | | |
| | Cool to 60° C. whilst mixing | | 100-60° C. | 30 min |
| 2 | Sorbitol (l) | 9.1% = 0.74 g | 60° C. | 15 min |
| | Sorbitol (s) | 21.25% = 1.73 g | | |
| | Microwax | 6% = 0.49 g | | |
| | Ibuprofen | 2% = 0.16 g | | |
| | Peppermint | 1% = 0.08 g | | |
| | flavour | | | |

The product extruded as a malleable white solid, and was shaped following the normal procedure.

Ibuprofen release from the gum containing P1, whose formulation is described in Example 7, was then compared with that from this control gum using the procedure described in Reference Example C. FIG. 4 displays the results. As will be apparent from the data, the gum with P1 has a significantly faster release of ibuprofen over the first 30 min (a realistic chewing time). It is not until 60 min that the total amount of nicotine released from the gums is similar.

### EXAMPLE 10

### Formulation of Gum using Nicotine Polacrilex

### Objective

To blend a chewing gum in which nicotine is introduced in the form of nicotine polacrilex. This consists of freebase nicotine adsorbed onto Amberlite IRP64 cation exchange resin. In the batch of polacrilex used in this example 21.5 weight percent of the material was nicotine. This polacrilex is often used in place of nicotine oil, as it is easier to handle and increases the stability of the nicotine. The finished gum produced in this example is expected to contain 2 mg of nicotine per gram of gum.

### Experimental

1 g P1 was mixed thoroughly with 0.154 g nicotine polacrilex in an aluminium vial, using a spatula. The nicotine/P1 mixture was blended into R3 gum base (Table 6) in the sweetener stage using the amended low temperature formulation, as described below.

TABLE 22
Formulation details for finished gum at lower temperature -
adjusted formulation

| Stage | Chemicals | Quantities | Temperature | Duration |
|---|---|---|---|---|
| 1 | Gum base | 39.0% = 3.10 g | 100° C. | 30 min |
| | Sorbitol (s) | 22.1% = 1.76 g | | |
| 2 | Cool to 60° C. | | 100-60° C. | 30 min |
| | whilst mixing | | | |
| 3 | P1 | 6.0% = 0.48 g | 60° C. | 30 min |
| | Nicotine polacrilex | 0.9% = 0.074 g | | |
| | Sorbitol (l) | 9.5% = 0.76 g | | |
| | Sorbitol (s) | 22.1% = 1.76 g | | |
| | Peppermint flavour | 1% = 0.08 g | | |

The gum extruded as a homogeneous white tape, which was then rolled between 2 glass surfaces to form 1 g cylinders of gum.

See Example 11 for a comparison of the nicotine release from this gum and a control gum without P1, made with an otherwise comparative formulation and methodology.

### EXAMPLE 11

### Formulation of Control Gum Containing Nicotine Polacrilex

### Objective

To blend a chewing gum control sample containing nicotine polacrilex, using microwax instead of P1 for comparison with Example 10. The finished gum produced in this Example is expected to contain 2 mg of nicotine per gram of gum.

### Experimental

1 g microwax was mixed thoroughly with 0.154 g nicotine polacrilex in an aluminium vial, using a spatula. A small amount of heating (to approx. 30° C.) was required to initially soften the wax. The nicotine/microwax mixture was then blended into S3 gum base (formulation in Table 7) in the sweetener stage using the amended low temperature formulation, as described below.

TABLE 23
Formulation details for finished gum at 60° C.

| Stage | Chemicals | Quantities | Temperature | Duration |
|---|---|---|---|---|
| 1 | Gum base | 39.0% = 3.10 g | 100° C. | 30 min |
| | Sorbitol (s) | 22.1% = 1.76 g | | |
| 2 | Cool to 60° C. | | 100-60° C. | 30 min |
| | whilst mixing | | | |
| 3 | Microwax | 6.0% = 0.48 g | 60° C. | 30 min |
| | Nicotine polacrilex | 0.9% = 0.074 g | | |
| | Sorbitol (l) | 9.5% = 0.76 g | | |
| | Sorbitol (s) | 22.1% = 1.76 g | | |
| | Peppermint flavour | 1% = 0.08 g | | |

The gum extruded as a white tape, and was rolled between two glass surfaces to form a cylindrical shape forming 1 g cylinders of gum.

FIG. 5 depicts accumulative nicotine release from this control gum and the comparative gum with P1 in artificial saliva determined using the method described in Reference Example C. The rate of release of nicotine from the gum with P1 is substantially higher than that from the control during much of the course of experiment. As a result, the total release of nicotine from the gum with amphiphilic polymer P1 increases substantially above that of the control at an early point in the experiment, and for the rest of the duration of the experiment. The total release from the gum with P1 is roughly twice that of the control after 60 min.

### EXAMPLE 12

### Formulation of Gum containing Caffeine

### Objective

To blend a chewing gum containing 47 mg of caffeine per gram of finished gum. The formulation includes P1 to control the speed of the release of the active ingredient. This gum varies principally from that described in example 3 in that the caffeine was mixed with P1 and added during the final stage of gum manufacture, as opposed to the earlier example where the P1 is instead incorporated into the gum base.

### Experimental

1 g P1 was mixed thoroughly with 0.78 g caffeine in an aluminium vial, using a spatula. The caffeine/P1 mixture was blended into R3 gum base (Table 5) with the sweeteners during the final stage of the manufacture of the formulation described in Table 24.

TABLE 24
Formulation details for P1 caffeine gum

| Stage | Chemicals | Ouantities | Temperature | Duration |
|---|---|---|---|---|
| 1 | Gum base | 37.5% = 3.00 g | 100° C. | 15 min |
| | Sorbitol (s) | 21.3% = 1.70 g | | |
| 2 | Cool to 60° C. | | 100-60° C. | 30 min |
| | whilst mixing | | | |
| 3 | P1 | 6.0% = 0.48 g | 60° C. | 30 min |
| | Caffeine | 4.7% = 0.38 g | | |
| | Sorbitol (l) | 9.1% = 0.73 g | | |
| | Sorbitol (s) | 21.3% = 1.70 g | | |
| | Peppermint flavour | 1% = 0.08 g | | |

The gum extruded as a homogeneous malleable white tape, which was then rolled between 2 glass surfaces to form 1 g cylinders of gum.

See Example 13 for a comparison of the caffeine release from this gum and a control gum without P1, made with an otherwise comparative formulation and methodology.

### EXAMPLE 13

### Formulation of Control Gum Containing Caffeine

### Objective

To blend a chewing gum sample containing caffeine without P1 for comparison with Example 12; microwax is used in the gum base in the place of P1. The finished gum produced in this Example is expected to contain 47 mg of caffeine per gram of gum.

### Experimental

The caffeine was blended into S3 gum base (formulation in Table 7) with the sweeteners during the final stage of the manufacture of the formulation described in Table 25.

TABLE 25
Formulation details for finished caffeine control gum

| Stage | Chemicals | Quantities | Temperature | Duration |
|---|---|---|---|---|
| 1 | Gum base | 40.0% = 3.21 g | 100° C. | 15 min |
| | Sorbitol (s) | 22.7% = 1.81 g | | |
| 2 | Cool to 60° C. | | 100-60° C. | 30 min |
| | whilst mixing | | | |
| 3 | Sorbitol (l) | 9.7% = 0.78 g | 60° C. | 30 min |
| | Sorbitol (s) | 22.7% = 1.81 g | | |
| | Caffeine | 4.7% = 0.38 g | | |
| | Peppermint flavour | 1% = 0.08 g | | |

The gum extruded as a malleable white tape, and was rolled between two glass surfaces to form a cylindrical shape forming 1 g cylinders of gum.

FIG. 6 depicts accumulative caffeine release from this control gum and the comparative gum with P1 in artificial saliva determined using the method described in Reference Example C. The rate of release of caffeine from the gum with P1 was equal or greater than that from the control during of the course of experiment. More specifically, caffeine release is observed to occur at a greater rate from the P1 containing gum for the duration of the first 20 min of the experiment. As a result, the total amount of caffeine released from the gum with amphiphilic polymer P1 was determined to be 16% higher than that from the control gum after the first data point (5 min), and was at least 20% higher than that of the control at every later data point.

### EXAMPLE 14

### Use of the Amphiphilic Graft Copolymers to Mediate the Release of a Chemical Entity from Chewing Gum

### 14.1 Aims

To demonstrate that the use of the graft copolymers in chewing gum in mediating the release of a chemical entity (in this case the commercial flavour cinnamaldehyde). Careful control of the release of flavour in medicated chewing gum is important to ensure that the taste associated with some active ingredients is masked.

### 14.2 Methodology

### Chemicals

Calcium carbonate (CaCO₃), ester gum, hydrogenated vegetable oil (HVO, hydrogenated soy oil), polyisobutylene (PIB, of molecular weight 51,000 g mol⁻¹), poly(vinyl acetate) (PVAc, of molecular weight 26,000 g mol⁻¹), glyceromonostearate (GMS), microcrystalline wax (microwax of m.p. 82-90° C.), sorbitol liquid, and sorbitol solid, were all food grade materials obtained from the Gum Base Company. Cinnamaldehyde (98+%) was obtained from Fisher-Scientific UK.

### Manufacture of Chewing Gum

The chewing gum base had the composition as shown in the table below:

TABLE 26
Recipe for the Manufacture of the Gum Bases; X is one of the
new graft copolymers, or microcrystalline wax in the case of
the S3 control; HVO = hydrogenated vegetable oil;
PVAc = poly(vinyl acetate).

| Stage | Component | % Composition | Mass/g |
|---|---|---|---|
| 1 | PIB | 13 | 1.04 |
| | PVAc | 6 | 0.48 |
| | CaCO₃ | 6 | 0.48 |
| | Ester Gum | 3.6 | 0.288 |
| 2 | Ester Gum | 5.4 | 0.432 |
| | CaCO₃ | 9 | 0.72 |
| 3 | PVAc | 9 | 0.72 |
| | Ester Gum | 9 | 0.72 |
| | CaCO₃ | 15 | 1.2 |
| 4 | HVO | 12 | 0.96 |
| | GMS | 6 | 0.48 |
| | X | 6 | 0.48 |
| | Total | 100 | 8 |

The gum base materials were mixed on a Haake Minilab micro compounder manufactured by the Thermo Electron Corporation, which is a small scale laboratory mixer/extruder. The ingredients were mixed together in four steps, the gum only being extruded after the final step. The gum base was mixed at 100° C.

The chewing gum was mixed according to the following table.

TABLE 27
Ingredients for the Chewing Gum X is one of the new graft
copolymers, or microcrystalline wax in the case of the S3 control.

| Stage | Time | Component | Amount | |
|---|---|---|---|---|
| 1 | 15 min | 37.5% Gum Base Containing X | 3 | g |
| | | 10% Sorbitol Liquid | 0.8 | g |
| | | 17% Sorbitol Powder | 1.36 | g |
| 2 | 15 min | 25.5% Sorbitol Powder | 2.04 | g |
| | | 6% X | 0.48 | g |
| | | 3% Sorbitol Liquid | 0.24 | g |
| | | 1% Cinnamaldehyde Flavour | 0.08 | mL |
| | 30 min | TOTAL | 8 | g |

The gum was mixed using the same equipment as the base and extruded after the final step. The gum was mixed at 60° C. In stage 1 the sorbitol liquid and powder were premixed prior to adding them to the gum.

### Experimental Method

See Reference Example D.

The samples were compared against standards (prepared in artificial saliva) covering the range 0.02-1.00 mg/mL. The retention time of cinnamaldehyde was determined to be 4.9 min on this equipment, thus the peak at this retention time was used to detect the released cinnamaldehyde. The samples were chewed two or three times, and in all cases two consistent release curves were generated. All of the samples were run in duplicate on the HPLC apparatus, indicating the results were highly reproducible.

### 14.3 Results

Gums have been made with polymers A-D and F-I, and chewed in artificial saliva, the released cinnamaldehyde is analyzed by HPLC. A control (S3) in which the graft copolymers were replaced with microwax was also made, and analyzed in the same manner (FIG. 7).

The control (S3) is observed to give a fairly steady release of cinnamaldehyde culminating in approximately 60% release after 60 min. Whilst two (H and I) graft copolymer containing gums have release profiles similar to the microwax material, most have either faster and higher maximum, or slower and lower maximum release profiles of the cinnamaldehyde. For instance, polymer H only releases 40% of the cinnamaldehyde in the gum after 60 min; compared with 50% in the case of the control. By contrast, cinnamaldehyde release from the gum made using D appears to have reached a plateau of approximately 70% cinnamaldehyde release before 30 min. The release rate from the gum containing C was slower, but the maximum release was comparable or slightly higher.

### 14.4 Conclusions

By altering the backbone and the degree of grafting (therefore hydrophilicity) of the amphiphile it is possible to alter the release profile of chemical species from chewing gum, in this case demonstrated with cinnamaldehyde. The release rate is seems to be determined by a number of factors including chemical identity of the backbone, and degree of grafting, resulting in changes in the interactions with saliva and other components of the gum. Therefore graft copolymer systems with a range of different release rates potentially available for formulation into chewing gum are disclosed.

### EXAMPLE 15

### Use of the Amphiphilic Graft Copolymers to Mediate the Release of an Active Ingredient

### 15.1 Aims

To demonstrate the use of the amphipihilic graft copolymers to deliver and release active ingredients, demonstrated by looking at the release of ibuprofen from solid mixtures of the polymers and ibuprofen, i.e. where the ibuprofen has been encapsulated. By encapsulated, we mean that the active ingredient is physically coated by, or encased, within the graft copolymer. Such an encapsulated material would then be dispersed in chewing gum using the methods described in this invention, in order to make it more palatable to the consumer.

### 15.2 Methodology

### Materials

Ibuprofen (40 grade) was obtained from Albemarle.

### Creation of Solid Mixes of Polymer and Ibuprofen

The powdered graft copolymer and ibuprofen were weighed out into a beaker to ensure that the ibuprofen comprised 1 weight percent. The two were premixed with a spatula to create a roughly homogenous mixture, and then mixed and extruded using the Haake Minilab micro compounder at 60° C. In the case of Polymer B 3.96 g of polymer and ibuprofen (0.04 g) were used; in the case of Polymer C 2.97 g of polymer and ibuprofen (0.03 g) were used.

### Testing Method

The encapsulated ibuprofen samples (approximately 1 g material of known weight) were placed between two plastic meshes and chewed mechanically in artificial saliva. Details of the mastication of the encapsulated ibuprofen is identical to that used with the cinnamaldehyde chewing gum above, samples being taken after 5 min, 10 min, 15 min, 20 min, 25 min, 30 min, 40 min, 50 min, and 60 min. Following this they were prepared for HPLC analysis by filtering them through a 10 mm PTFE acrodisc syringe filter. The samples were analyzed using the HPLC apparatus described in Reference Example D.

The encapsulated ibuprofen samples were chewed two or three times, and in all cases two consistent release curves were generated. All of the samples were run in duplicate on the HPLC apparatus, indicating the results were highly reproducible.

### 15.3 Results

Two different polymers were used to encapsulate the ibuprofen, both were chewed and the release profile monitored by HPLC (FIG. 8).

Both of the polymer/ibuprofen mixtures released ibuprofen into solution during chewing, and released similar total amounts of ibuprofen into the saliva—around 60% of the maximum total, a point at which the release seems to plateau in the two examples tested. Interestingly the release of ibuprofen is much more rapid in the case of polymer B than polymer C, whereas both polymers have chemically similar backbones, the amount of MPEG grafted to the backbone is much higher in the case of B. A possible-explanation therefore is that increasing the hydrophilicity of the polymers aids disintegration of the encapsulated samples, resulting in faster release during chewing/grinding (the polymers are hard solids).

### 15.4 Conclusions

Ibuprofen was encapsulated in two samples of the graft copolymers, and released by masticating the samples in artificial saliva. Graft copolymer B releases ibuprofen more rapidly than graft copolymer C, the former also contains more PEG and is more hydrophilic. It seems that by adjusting the hydrophilicity of the amphiphilic graft copolymers it is possible to alter the release rate of the ibuprofen.

1. A chewing gum composition comprising a chewing gum base, a biologically active ingredient, a polymeric material and one or more sweetening or flavouring agents, wherein the polymeric material is amphiphilic, has a straight or branched chain carbon-carbon backbone and a multiplicity of side chains attached to the backbone.

2. A chewing gum composition according to claim 1, wherein the backbone of the said polymeric material is derived from a homopolymer of an ethylenically unsaturated hydrocarbon monomer or from a copolymer of two or more ethylenically-unsaturated polymerisable hydrocarbon monomers, and the side chains are hydrophilic.

3. A chewing gum composition according to claim 1, wherein the carbon-carbon polymer backbone is derived from a homopolymer of an ethylenically-unsaturated polymerisable hydrocarbon monomer containing 4 or 5 carbon atoms.

4. A chewing gum composition according to claim 3, wherein the carbon-carbon polymer backbone is derived from a homopolymer of isobutylene, butadiene or isoprene.

5. A chewing gum composition according to claim 1, wherein the side chains of the polymeric material are derived from polyethylene oxide), polyglycine, poly(vinyl alcohol), poly(styrene sulphonate) or poly(acrylic acid).

6. A chewing gum composition according to claim 1, wherein the side chains are attached to the backbone via groups derived from malefic anhydride.

7. A chewing gum composition according to claim 1, wherein the polymeric material has pendant carboxylic acid groups.

8. A chewing gum composition according to claim 1, wherein the backbone of the amphiphilic polymeric material has a molecular weight in the range 10,000-200,000.

9. A chewing gum composition according to claim 1, wherein the ratio of side chains to backbone units is in the range 1:350 to 1:20.

10. A chewing gum composition according to claim 1, wherein the side chains have the formula 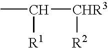
wherein R¹ is H, —C(O)OR⁴ or —C(O)Q and R² is —C(O)OR⁴ or —C(O)Q provided that at least one of R¹ and R² is the group —C(O)Q;
R³ is H or —CH₃;
R⁴ is H or an alkyl group having from 1 to 6 carbon atoms;
Q is a group having the formula —O—(YO)_{b}—(ZO)_{c}—R⁵, wherein each of Y and Z is, independently, an alkylene group having from 2 to 4 carbon atoms and R⁵ is H or an alkyl group having from 1 to 4 carbon atoms;
each of b and c is, independently, 0 or an integer of from 1 to 125 provided that the sum b+c has a value in the range of from 10 to 250.

11. A chewing gum composition according to claim 10, wherein the side chains in the polymeric material have the formula 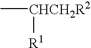
wherein one of R¹ and R² is —C(O)Q and the other is —C(O)OR⁴, in which R⁴ is H or an alkyl group having from 1 to 6 carbon atoms;
Q is a group having the formula —O—(YO)_{b}—(ZO)_{c}—R⁵ wherein each of Y and Z is, independently, an alkylene group having from 2 to 4 carbon atoms and R⁵ is H or an alkyl group having from 1 to 4 carbon atoms;
each of b and c is, independently, 0 or an integer of from 1 to 125 provided that the sum b+c has a value in the range of from 10 to 250.

12. A chewing gum composition according to claim 1, wherein the biologically active ingredient is a pharmaceutically active ingredient.

13. A chewing gum composition according to claim 1, wherein the biologically active ingredient is selected from anti-platelet aggregation drugs, erectile dysfunction drugs, decongestants, anaesthetics, oral contraceptives, cancer chemotherapeutics, psychotherapeutic agents, cardiovascular agents, NSAID's, NO Donors for angina, non-opioid analgesics, antibacterial drugs, antacids, diuretics, anti-emetics, antihistamines, anti-inflammatories, antitussives, anti-diabetic agents, opioids, and hormones and combinations thereof.

14. A chewing gum composition according to claim 13, wherein the biologically active ingredient is a stimulant.

15. A chewing gum composition according to claim 13, wherein the biologically active ingredient is a non-steroidal anti-inflammatory drug, such as diclofenac, ketoprofen, ibuprofen, aspirin or naproxen.

16. A chewing gum composition according to claim 13, wherein the biologically active ingredient is a vitamin, mineral or other nutritional supplement.

17. A chewing gum composition according to claim 1, wherein the chewing gum base comprises an elastomeric material other than the polymeric material, elastomer plasticiser, a softener, filler, an emulsifier and optionally wax.

18. A chewing gum composition according to claim 1, wherein the chewing gum base comprises the said polymeric material.

19. A chewing gum composition according to claim 1, which comprises 0.1-50% said polymeric material.

20. A chewing gum composition according to claim 1, wherein the chewing gum composition is in the form of a unit suitable for oral administration, and comprises 1-400 mg of biologically active ingredient.

21. A chewing gum composition according to claim 20, wherein the biologically active ingredient is nicotine and the unit of composition comprises 1-5 mg of nicotine.

22. A chewing gum composition according to claim 20, wherein the active ingredient is a non-steroidal anti-inflammatory drug, and the unit of composition comprises 10 mg-100 mg drug.

23. A method of forming a chewing gum composition comprising the steps of: (i) forming a chewing gum base by mixing an elastomeric material optionally with one or more elastomer plasticisers, softeners, fillers, emulsifiers and waxes; and (ii) adding the biologically active ingredient to the gum base together with one or more sweetening or flavouring agents, to form a chewing gum composition;
wherein a polymeric material which is amphiphilic and has a straight or branched chain carbon-carbon backbone and a multiplicity of side chains attached to the backbone, is added to the chewing gum base in step (i), and/or to the chewing gum composition in step (ii).

24. A method according to claim 23 wherein in step (ii), the gum base is heated.

25. A method according to claim 24 wherein in step (ii), after heating, the mixture is cooled and the biologically active ingredient added to the cooled mixture.

26. A method according to claim 23, wherein the chewing gum composition is extruded after step (ii) shaped to form a unit chewing gum composition.

27. A method according to claim 23, comprising a preliminary step, wherein the biologically active ingredient is mixed with the polymeric material or a sweetening agent before being added to the chewing gum base in step (ii).

28. A method of forming a chewing gum composition comprising the steps of: (i) forming a chewing gum base by mixing an elastomeric material optionally with one or more elastomer plasticisers, softeners, fillers, emulsifiers and waxes; and (ii) adding the biologically active ingredient to the gum base together with one or more sweetening or flavouring agents, to form a chewing gum composition;
wherein a polymeric material which is amphiphilic and has a straight or branched chain carbon-carbon backbone and a multiplicity of side chains attached to the backbone, is added to the chewing gum base in step (i), and/or to the chewing gum composition in step (ii),
wherein the chewing gum composition has the features of claim 1.
